(19) United States
(12) Patent Application Publication
(10) Pub. No.: US 2010/0063020 A1
(43) Pub. Date: Mar. 11, 2010

(54) ORAL PHARMACEUTICAL FORMULATIONS AND METHODS FOR PRODUCING AND USING SAME

(75) Inventors: Robert K. Mansfield, Carlsbad, CA (US); Edgar H. Ulm, Encinitas, CA (US); Gregg A. Timony, San Diego, CA (US)

Correspondence Address: BIOGEN IDEC / FINNEGAN HENDERSON, LLP, 901 NEW YORK AVENUE, NW, WASHINGTON, DC, 20001-4413, US

(73) Assignee: CONFORMA THERAPEUTICS CORPORATION

(21) Appl. No.: 12/554,832

(22) Filed: Sep. 4, 2009

Related U.S. Application Data
(63) Continuation of application No. 10/954,994, filed on Sep. 29, 2004.

(51) Int. Cl. A61K 31/395; A61P 31/00

(52) U.S. Cl. 514/183

(57) ABSTRACT

Oral pharmaceutical formulations and methods of producing and using the same are described and claimed. The formulations are dispersions of phospholipids and one or more pharmacologically active compounds, In preferred embodiments, the pharmaceutically active compounds are ansamycins, pharmaceutically acceptable salts, or prodrugs thereof.

### RELATED APPLICATIONS

This application claims priority to International Patent Application Serial Number PCT/US03/31667, entitled DRUG FORMULATIONS HAVING LONG AND MEDIUM CHAIN TRIGLYCERIDES, filed Oct. 4, 2003, which claims priority to U.S. Provisional Patent Application Ser. No. 60/491,050, filed Jul. 29, 2003 and entitled ANSAMYCIN FORMULATIONS AND METHODS FOR PRODUCING AND USING SAME; U.S. Provisional Patent Application Ser. No. 60/478,430, filed Jun. 12, 2003 and entitled PHOSPHOLIPID-BASED FORMULATIONS AND METHODS FOR PRODUCING AND USING SAME; U.S. Provisional Patent Application Ser. No. 60/454,812, filed Mar. 13, 2003 and entitled HSP90-INHIBITOR FORMULATIONS AND DATA, and International Patent Application Serial Number PCT/US03/10533, filed Apr. 4, 2003 and entitled ANSAMYCIN FORMULATIONS AND METHODS FOR PRODUCING AND USING SAME, which in turn claims priority to U.S. Provisional Application Ser. No. 60/371,668, filed Apr. 10, 2002 and entitled NOVEL ANSAMYCIN FORMULATIONS AND METHODS FOR PRODUCING AND USING SAME. Each of the above cited U.S. Provisional Applications and International Applications is expressively incorporated herein by reference in its entirety.

### FIELD OF INVENTION

The invention relates in general to oral pharmaceutical formulations and methods of producing and using the same; more particularly, the invention relates to oral formulations of ansamycins, e.g., 17-allylamino-geldanamycin (17-AAG).

### BACKGROUND

Ansamycins are antibiotic molecules characterized by an “ansa” structure which comprises any one of benzoquinone, benzohydroquinone, naphthoquinone or naphthohydroquinone moieties bridged by a long chain. Geldanamycin (GDM) and its synthetic semi-synthetic analog 17-allylamino-geldanamycin (17-AAG) belong to the benzoquinone class of ansamycins. GDM, as first isolated from the microorganism Streptomyces hygroscopicus, was originally identified as a potent inhibitor of certain kinases, and was later shown to act by stimulating kinase degradation, specifically by targeting “molecular chaperones,” e.g., heat shock protein 90s (HSP90s). Subsequently, various other ansamycins have demonstrated more or less such activity, with 17-AAG being among the most promising and the subject of intensive clinical studies currently being conducted by the National Cancer Institute (NCI). See, e.g., Federal Register, 66(129): 35443-35444; Erlichman et al., Proc. AACR 2001, 42, abstract 4474.

HSP90s are ubiquitous chaperone proteins involved in folding, activation and assembly of a wide range of proteins, including key proteins involved in signal transduction, cell cycle control and transcriptional regulation. Researchers have reported that HSP90 chaperone proteins are associated with important signaling proteins, such as steroid hormone receptors and protein kinases, including, e.g., Raf-1, EGFR, v-Src family kinases, Cdk4, and ErbB-2 (Buchner J., 1999, TIBS, 24:136-141; Stepanova, L. et al., 1996, Genes Dev. 10:1491-502; Dai, K. et al., 1996, J Biol. Chem. 271:22030-4). Studies further indicate that certain co-chaperones, e.g., HSP70, p60/Hop/Sti1, Hip, Bag1, HSP40/Hdj2/Hsj1, immunophilins, p23, and p50, may assist HSP90 in its function (see, e.g., Caplan, A., Trends in Cell Biol., 9: 262-68 (1999)).

Ansamycin antibiotics, e.g., herbimycin A (HA), geldanamycin (GDM), and 17-AAG, are thought to exert their anticancerous effects by tight binding of the N-terminus ATP-binding pocket of HSP90 (Stebbins, C. et al., 1997, Cell, 89:239-250). This pocket is highly conserved and has weak homology to the ATP-binding site of DNA gyrase (Stebbins, C. et al., supra; Grenert, J. P. et al., J. Biol. Chem. 1997, 272:23843-50). Further, ATP and ADP have both been shown to bind this pocket with low affinity and to have weak ATPase activity (Proromou, C. et al., Cell, 1997, 90: 65-75; Panaretou, B. et al., EMBO J., 1998, 17: 482936). In vitro and in vivo studies have demonstrated that occupancy of this N-terminal pocket by ansamycins and other HSP90 inhibitors alters HSP90 function and inhibits protein folding. At high concentrations, ansamycins and other HSP90 inhibitors have been shown to prevent binding of protein substrates to HSP90 (Scheibel, T., H. et al., 1999, Proc. Natl. Acad. Sci. USA 96:1297-302; Schulte, T. W. et al., 1995, J. Biol. Chem. 270:24585-8; Whitesell, L., et al., 1994, Proc. Natl. Acad. Sci. USA 91:8324-8328). Ansamycins have also been demonstrated to inhibit the ATP-dependent release of chaperone-associated protein substrates (Schneider, C., L. et al., 1996, Proc. Natl. Acad. Sci. USA, 93:14536-41; Sepp-Lorenzino et al., 1995, J. Biol. Chem. 270:16580-16587). In either event, the substrates are degraded by a ubiquitin-dependent process in the proteasome (Schneider, C., L., supra; Sepp-Lorenzino, L., et al., 1995, J. Biol. Chem., 270:16580-16587; Whitesell, L. et al., 1994, Proc. Natl. Acad. Sci. USA, 91: 8324-8328).

This substrate destabilization occurs in tumor and non-transformed cells alike and has been shown to be especially effective on a subset of signaling regulators, e.g., Raf (Schulte, T. W. et al., 1997, Biochem. Biophys. Res. Commun. 239:655-9; Schulte, T. W., et al., 1995, J. Biol. Chem. 270:24585-8), nuclear steroid receptors (Segnitz, B., and U. Gehring. 1997, J. Chem. 272:18694-18701; Smith, D. F. et al., 1995, Mol. Cell. Biol. 15:6804-12), v-src (Whitesell, L., et al., 1994, Proc. Natl. Acad. Sci. USA 91:8324-8328) and certain transmembrane tyrosine kinases (Sepp-Lorenzino, L. et al., 1995, J. Biol. Chem. 270:16580-16587) such as EGF receptor (EGFR), Her2/Neu (Hartmann, F. et al., 1997, Int. J. Cancer 70:221-9; Miller, P. et al., 1994, Cancer Res. 54:2724-2730; Mimnaugh, E. G. et al., 1996, J. Biol. Chem. 271:22796-801; Schnur, R. et al., 1995, J. Med. Chem. 38:3806-3812), CDK4, and mutant p53 (Erlichman et al., Proc. AACR 2001, 42, abstract 4474). The ansamycin-induced loss of these proteins leads to the selective disruption of certain regulatory pathways and results in growth arrest at specific phases of the cell cycle (Muise-Heimericks, R. C. et al., 1998, J. Biol. Chem. 273:29864-72), and apoptsosis, and/or differentiation of cells so treated (Vasilevskaya, A. et al., 1999, Cancer Res., 59:3935-40).

In addition to anti-cancer and antitumorgenic activity, HSP90 inhibitors have also been implicated in a wide variety of other utilities, including use as anti-inflammation agents, anti-infectious disease agents, agents for treating autoimmunity, agents for treating stroke, ischemia, cardiac disorders and agents useful in promoting nerve regeneration (See, e.g., Rosen et al., PCT Publication No. WO 02/09696 (PCT/USO1/23640); Degranco et al., WO 99/51223 (PCT/US99/07242); Gold, U.S. Pat. No. 6,210,974 B1; DeFranco et al., U.S. Pat. No. 6,174,875). Overlapping somewhat with the above, there are reports in the literature that fibrogenetic disorders including but not limited to scleroderma, polymyositis, systemic lupus, rheumatoid arthritis, liver cirrhosis, keloid formation, interstitial nephritis, and pulmonary fibrosis also may be treatable. (Strehlow, PCT Publication No. WO 02/02123 (PCT/US01/20578)). Still further HSP90 modulation, modulators and uses thereof are reported in International Application Nos. PCT/US03/04283, PCT/US02/35938, PCT/US02/16287, PCT/US02/06518, PCT/US98/09805, PCT/US00/09512, PCT/US01/09512, PCT/US01/23640, PCT/US01/46303, PCT/US01/46304, PCT/US02/06518, PCT/US02/29715, PCT/US02/35069, PCT/US02/35938, PCT/US02/39993, PCT/US03/10533, PCT/US03/02686, and U.S. Provisional Application Nos. 60/293,246, 60/371,668, 60/331,893, 60/335,391, 06/128,593, 60/337,919, 60/340,762, and 60/359,484.

The administration route which has the highest patient compliance for a drug, particularly one which requires persistent dosing such as an antiproliferative drug, is by an oral route. Such route of administration, which may be by means of syrup, elixir, tablets, capsules, granules, powders or any other convenient formulation, is generally simple and straightforward and is frequently the least inconvenient or unpleasant from the patient's point of view. Ansamycins like many other lipophilic drugs are difficult to prepare for pharmaceutical applications. Particularly, several important ansamycins, e.g., 17-AAG, that are extremely water insoluble and the low dissolution rate made them unfavorable for oral dosing. Egorin et al. showed that, using an aqueous reconstituted lyophilized preparation containing sucrose, 17-AAG displays a 24% oral bioavailability. (See Egorin et al., Cancer Chemother Pharmacol 2002, 49, 7-19). Other researchers had concluded that it was not possible to give 17-AAG orally. (Kaur et al., Clinical Cancer Research 2004, 10, 4813-4821).

Accordingly, there exists a need for an oral formulation for ansamycins, the present invention addresses that need.

### SUMMARY OF THE INVENTION

The invention features pharmaceutical formulations and compositions of ansamycins, particularly 17-AAG, which are suitable for oral administration.

In one aspect, the invention features a pharmaceutical formulation for oral administration which includes a pharmaceutically effective amount of an ansamycin and one or more pharmaceutically acceptable solubilizers. The solubilizer is a phospholipid, sodium lauryl sulfate, Tween 80, polyethylene glycols, Solutol HS15, or combinations thereof; dimethylsulfoxide (DMSO) is expressly excluded as a solubilizer for the formulations of the invention. The pharmaceutical formulation may further include a co-solvent such as glycerol, Labrafil, Labrasol, polyethylene glycols of various molecular weights, Tween 80, Tween 20, and Solutol HS15, propylene carbonate, ethanol, Transcutol HP, glycofurol, oleic acid, short-, medium- or long-chain triglycerides, synthetic and non-synthetic surfactants, and combinations thereof. In addition, the pharmaceutical formulation may further include other excipients such as lubricants, glidants, fillers, wetting agents, binders, disintegrants, flavoring agents, suspending agents, and combinations thereof.

The ansamycin in the pharmaceutical formulation is a member selected from the group below, or a polymorph, solvate, ester, tautomer, enantiomer, pharmaceutically acceptable salt or prodrug thereof:

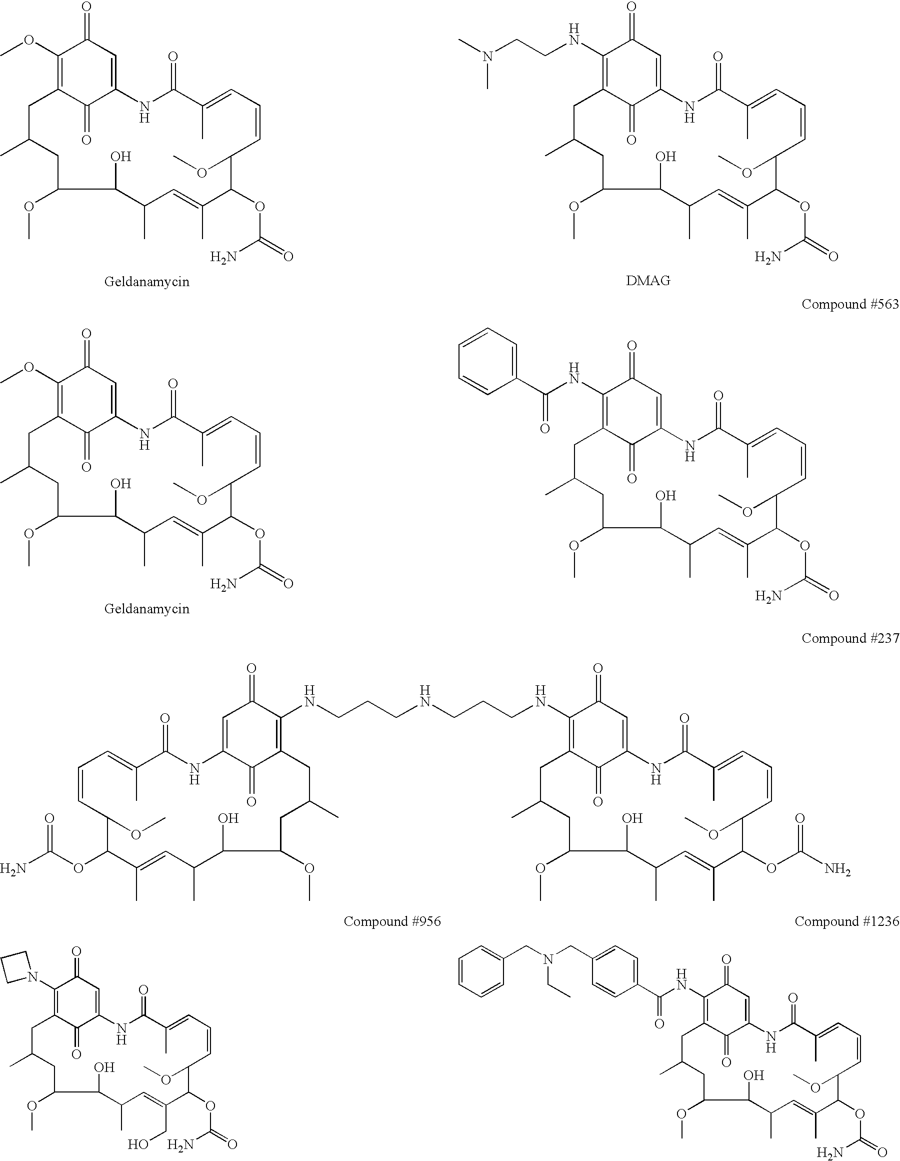

In one embodiment, the ansamycin is 17-AAG. In another embodiment, the ansamycin is a mixture of low melt forms of 17-AAG which is characterized by DSC melting temperatures at below 175° C. and by an X-ray powder diffraction pattern having three peaks located at 5.85, 4.35 and 7.90 two-theta angles. In another embodiment, the ansamycin is a low melt polymorph of 17-AAG which is characterized by a DSC melting temperature at about 156° C. and by an X-ray powder diffraction pattern having three peaks located at 5.85 degree, 4.35 degree and 7.90 degree two-theta angles. In yet another embodiment, the ansamycin is another low melt polymorph of 17-AAG characterized by a DSC melting temperature at about 172° C.

In one embodiment, the solubilizer is a phospholipid, such as Phospholipon 90G, phosphatidylcholine, phosphatidylserine, phosphatidylinositol or phosphatidylethanolamine, and the phospholipid is present in a concentration of at least 5% w/w of the formulation and at most 75% w/w of the formulation. In one embodiment, the phospholipid is present at about 10% to about 50%. In another embodiment, the solubilizer of the pharmaceutical formulation is sodium lauryl sulfate which is present at about 0.5% to about 50% w/w. In yet another embodiment, the solubilizer is a mixture of polyethylene glycol (PEG) and Tween 80, and the ratio of PEG to Tween 80 in the mixture can be 4:1 w/w.

Another aspect of the invention is an oral pharmaceutical composition which includes a pharmaceutically effective amount of an ansamycin and one or more excipients, in which at least one of the excipients is a phospholipid present in a concentration of at least 5% w/w of the composition. The ansamycin in this phospholipid oral pharmaceutical composition is a member selected from the group of ansamycins described above.

In one embodiment of the phospholipid oral pharmaceutical composition, the ansaymycin is 17-AAG. The 17-AAG may be a high melt 17-AAG, a low melt 17-AAG, an amorphous form of 17-AAG, or combinations thereof. In one embodiment, the ansamycin is a low melt 17-AAG. The low melt 17-AAG may comprise polymorphs characterized by DSC melting temperatures below 175° C. and by an X-ray powder diffraction pattern having three peaks located at 5.85, 4.35 and 7.90 two-theta angles. The low melt 17-AAG may also comprise a polymorph that is characterized by a DSC melting temperature at about 156° C. and by an X-ray powder diffraction pattern having three peaks located at 5.85 degree, 4.35 degree and 7.90 degree two-theta angles. Further, the low melt 17-AAG may also comprise a polymorph that is characterized by a DSC melting temperature at about 172° C.

The oral bioavailability of the ansamycin in the phospholipid oral pharmaceutical composition, in one embodiment, is greater than 25%. In another embodiment, it is greater than 35%. In yet another embodiment, it is greater than 50%. In a further embodiment, it is greater than 60%.

The phospholipid in the oral pharmaceutical composition is a phosphoglyceride, sphinogomyelin or combinations thereof. In one embodiment, the phospholipid is phosphatidylcholine, phosphatidylserine, phosphatidylinositol, phosphatidylethanolamine, or Phospholipon 90G. The concentration of the phospholipid is 5% to about 75%. In one embodiment, the concentration of the phospholipid is about 10% to about 50%. In another embodiment, the concentration of the phospholipid is about 10% to about 25%.

The oral pharmaceutical composition may further include a co-solvent that is selected from the group consisting of glycerol, Labrafil, Labrasol, polyethylene glycol, Tween 80, Solutol HS15, propylene carbonate, Transcutol HP and glycofurol. The oral pharmaceutical composition may further includes other excipients selected from the group of lubricants, glidants, fillers, wetting agents, binders, disintegrants, flavoring agents, suspending agents, and combinations thereof.

In one embodiment of the oral pharmaceutical composition, the ansamycin is a low melt 17-AAG which has a bioavailability greater than 25%, and the phospholipid is Phospholion 90G which is present at about 10% to about 50% w/w of the composition. Further, the composition may include Tween 80.

In another aspect, the invention provides an oral pharmaceutical composition which includes a pharmaceutically effective amount of an ansamycin and one or more excipients, wherein at least one of the excipients is a non-lipid solublizer. The ansamycin in this non-lipid composition is selected from the group described above.

In one embodiment, the non-lipid solubilizer is selected from a group consisting of sodium lauryl sulfate, Tween 80, polyethylene glycols of various molecular weights, Labrasol, propylene carbonate, ethanol, Solutol HS15, Transcutol HP, glycofurol, oleic acid and short-, medium- or long-chain triglycerides, other synthetic and non-synthetic surfactants, and combinations thereof; dimethylsulfoxide (DMSO) is expressly excluded as a suitable solubilizer for the invention. In one embodiment, the non-lipid solubilizer is sodium lauryl sulfate (SLS) which is present in a concentration of about 0.5 to about 50% w/w of the composition. In one embodiment, the composition further includes Neusilin.

In another embodiment, the non-lipid solubilizer is a mixture of polyethylene glycol (PEG) and Tween 80 wherein the ratio of PEG to Tween 80 is 4:1 w/w. The oral bioavailability of the ansamycin in this PEG-Tween 80 composition is at least 25%.

In one embodiment of this non-lipid pharmaceutical composition, the ansamycin is 17-AAG. The 17-AAG can be a high melt form, a low melt form, an amorphous form, or combinations thereof. In one embodiment, the 17-AAG is a low melt form that is characterized by a DSC melting temperature of about 156° C. and by an X-ray powder diffraction pattern having three principal peaks located at 5.85, 4.35 and 7.90 two-theta angles. In another embodiment the 17-AAG is a low melt form which is characterized by a DSC melting temperature of about 171° C. In yet another embodiment, the 17-AAG includes low melt forms of 17-AAG which are characterized by DSC melting temperatures below 175° C. and by an X-ray powder diffraction pattern having three principal peaks located at 5.85, 4.35 and 7.90 two-theta angles.

The non-lipid pharmaceutical composition may include co-solvents selected from the group consisting of glycerol, Labrafil, Labrasol, polyethylene glycols of various molecular weights, Tween 80, Tween 20, and Solutol HS15, propylene carbonate, ethanol, Transcutol HP, glycofurol, oleic acid, short-, medium- or long-chain triglycerides, other synthetic and non-synthetic surfactants, and combinations thereof.

In another aspect, the invention provides an oral pharmaceutical composition comprising a pharmaceutical effective amount of a low melt 17-AAG and one or more pharmaceutically acceptable excipients and optionally co-solvents.

In one embodiment of the low melt 17-AAG oral pharmaceutical composition, the low melt 17-AAG is a form that is characterized by a DSC melting temperature of about 156° C. and by an X-ray powder diffraction pattern having peaks located at 5.85, 4.35 and 7.90 two-theta angles. In another embodiment the low melt 17-AAG is a form that is characterized by a DSC melting temperature of about 171° C. In yet another embodiment, the low melt 17-AAG includes more than one low melt form of 17-AAG characterized by DSC melting temperatures below 175° C. and by an X-ray powder diffraction pattern having three principal peaks located at 5.85, 4.35 and 7.90 two-theta angles.

In one embodiment of the low melt 17-AAG oral pharmaceutical composition, one of the excipient is a phospholipid present in a concentration between 5% to about 75% w/w of the composition. In another embodiment, the concentration of the phospholipid is between about 10% to 50% w/w of the composition. In another embodiment, the phospholipid is selected from phosphatidylcholine, phosphatidylserine, phosphatidylethanolamine, phosphatidylinositol, phosphatidylglycerol and Phospholipon 90G. In another embodiment, the phospholipid is Phospholipon 90G. The bioavailability of the 17-AAG is at least 25%, or at least 35%.

In another embodiment of the low melt 17-AAG oral pharmaceutical composition, the excipient is sodium lauryl sulfate.

In another embodiment of the low melt 17-AAG oral pharmaceutical composition, the excipient is a mixture of polyethylene glycol (PEG) and Tween 80. In one embodiment, the ratio of PEG to Tween is 80 to 20 w/w. In other embodiment, the 17-AAG has a bioavailability greater than 25%.

The above aspects and embodiments may be combined when feasible or appropriate. Other aspects and variation of the forgoing aspects and embodiments which are obvious to those skilled in the art are within the contemplation of the invention.

Advantages of the invention include one or more of ease of manufacture, the use of clinically acceptable reagents (e.g., having reduced environment and/or patient toxicity), enhanced formulation stability, less complicated shipping and warehousing, and simplified pharmacy and bed-side handling. Other advantages, aspects, and embodiments will be apparent from the description above and the detailed description and claims to follow.

### BRIEF DESCRIPTION OF THE DRAWINGS

FIG. 1 shows the X-ray powder diffraction pattern of the high melt form of 17-AAG showing three peaks at 7.40, 6.08 and 11.84 two-theta angles.

FIG. 2 shows the X-ray powder diffraction pattern of the low melt form of 17-AAG showing three peaks at 5.85, 4.35 and 7.90 two-theta angles.

FIG. 3 shows a differential scanning calorimetry (DSC) scan of the high melt form of 17-AAG.

FIG. 4 shows a differential scanning calorimetry (DSC) scan of the low melt form of 17-AAG.

FIG. 5 shows the intrinsic dissolution rate (mg/cm²) of low melt and high melt 17-AAG versus time (min) in ethanol.

FIG. 6 illustrates the effect of sodium lauryl sulfate (SLS) on the aqueous solubility of 17-AAG at pH 6.0.

FIG. 7 shows the serum concentrations of 17-AAG (mean±standard deviation) in rats following oral (50 mg/kg) or intravenous (10 mg/kg) administration of 17-AAG phospholipid complex.

FIG. 8 shows the serum concentrations of the metabolite 17-aminogeldanamycin (17-AG) (mean±standard deviation) in rats following oral (50 mg/kg) or intravenous (10 mg/kg) administration of a low melt 17-AAG lipid complex.

FIG. 9 shows the serum concentrations of 17-AAG (mean±standard deviation) and the metabolite 17-AG in rats following a mean dose of 14.3 mg/kg via oral administration of 17-AAG gel capsule. The graph symbols represent the mean serum concentration detected in three mice at each time point.

FIG. 10 shows the concentration of 17-AAG detected in serum of Balb/C mice given a 40 mg/kg dose of 17-AAG I.V. (▪) orally in lipid complex (), or I.P. (▴). The graph symbols represent the mean serum concentration detected in three mice at each time point.

FIG. 11 shows the concentration of 17-AAG detected in serum of Balb/C mice given a 40 mg/kg dose of 17-AAG I.V. (▪), orally in PEG-Tween (), or I.P. (▴). Graph symbols represent the mean serum concentration detected in three mice at each time point.

### DETAILED DESCRIPTION OF THE INVENTION

The invention features oral pharmaceutical formulations and methods of producing same. Applicants have observed that many of the ansamycins, e.g. 17-AAG, have poor water solubility/slow dissolution, but have excellent absorption properties. Applicants further observed that different polymorphic forms of crystalline ansamycins have different dissolution characteristics, e.g., 17-AAG has a low melt form which exhibits significantly higher dissolution rate than the high melt form. Taking advantage of these properties, Applicants have devised formulations which render water-insoluble drugs, e.g., ansamycins, suitable for oral administration to a patient. This is accomplished by selecting a formulation that affords immediate release of the drug from the dosage form in GI fluids once the compound is delivered and selecting a high dissolution polymorph of the drug to ensure rapid dissolution. The method of formulation is relatively simple, typically utilizes clinically acceptable reagents, and results in a product that affords storage stability. In the oral formulations, Applicants employ phospholipids and other non-lipid solubilizers to assist the dissolution of the ansamycins from the dose form. The formulated drug solution may then be combined with other excipients and processed into various forms for oral administration.

While the invention is illustrated using 17-AAG, it should be understood that the novel method of drug formulation described herein applied to both the high melt and low melt forms of the compound, and its polymorphs, tautomers, enantiomers, pharmaceutically acceptable salts, and prodrugs. It should be further understand that the method further applies to many other ansamycins including, but are not limited to, those exemplified in Examples 1-13 of the EXAMPLE section, such as geldanamycin, 17-N,N-dimethylaminoethylaminogeldanamycin, and polymorphs, tautomers, enantiomers, pharmaceutically acceptable salts, and prodrugs thereof. The structures of the numbered compounds are disclosed in the Summary section.

### I. Definitions

The following claim terms have the following meanings, and claim terms not specifically appearing below have their common customary meaning as used in the art:

The term “pharmacologically active compound”, “active pharmaceutical ingredient” or “therapeutical ingredient” is synonymous with “drug” and means any compound that exerts, directly or indirectly, a biological effect, in vitro or in vivo when administered to cultured cells or to an organism. The drug is preferably capable of encasement in liposomes and/or emulsification, and will typically, although not necessarily, be lipophilic.

A “prodrug” is a drug covalently bonded to a carrier wherein release of the drug occurs in vivo when the prodrug is administered to a mammalian subject. Prodrugs of the compounds of the present invention are prepared by modifying functional groups present in the compounds in such a way that the modifications are cleaved, either in routine manipulation or in vivo, to yield the desired compound. Prodrugs include compounds wherein hydroxy, amine, or sulfhydryl groups are bonded to any group that, when administered to a mammalian subject, is cleaved to form a free hydroxyl, amino, or sulfhydryl group, respectively. Examples of prodrugs include, but are not limited to, acetate, formate, or benzoate derivatives of alcohol or amine functional groups in the compounds of the present invention; phosphate esters, dimethylglycine esters, aminoalkylbenzyl esters, aminoalkyl esters or carboxyalkyl esters of alcohol or phenol functional groups in the compounds of the present invention; or the like. Prodrugs can impart multiple advantages for drug delivery, e.g., as explained in REMINGTON PHARMACEUTICAL SCIENCES, 20th Edition, Ch. 47, pp. 913-914.

“Pharmaceutically acceptable salts” include those derived from pharmaceutically acceptable inorganic and organic acids and bases. Examples of suitable acids include hydrochloric, hydrobromic, sulfuric, nitric, perchloric, fumaric, maleic, phosphoric, glycolic, gluconic, lactic, salicylic, succinic, toluene-p-sulfonic, tartaric, acetic, citric, methanesulfonic, formic, benzoic, malonic, naphthalene-2-sulfonic, benzenesulfonic, 1,2 ethanesulfonic acid (edisylate), galactosyl-d-gluconic acid and the like. Other acids, such as oxalic acid, while not themselves pharmaceutically acceptable, may be employed in the preparation of salts useful as intermediates in obtaining the compounds of this invention and their pharmaceutically acceptable acid addition salts. Salts derived from appropriate bases include alkali metal (e.g., sodium), alkaline earth metal (e.g., magnesium), ammonium and N—(C₁-C₄ alkyl)₄⁺ salts, and the like. Illustrative examples of some of these include sodium hydroxide, potassium hydroxide, choline hydroxide, sodium carbonate, and the like. Where the claims recite “a compound (e.g., compound ‘x’) or pharmaceutically acceptable salt thereof,” and only the compound is displayed, those claims are to be interpreted as embracing, in the alternative or conjunctive, a pharmaceutically acceptable salt or salts of such compound.

The term “ansamycin” is a broad term which characterizes compounds having an “ansa” structure which comprises any one of benzoquinone, benzohydroquinone, naphthoquinone or naphthohydroquinone moities bridged by a long chain. Compounds of the naphthoquinone or naphthohydroquinone class are exemplified by the clinically important agents rifampicin and rifamycin, respectively. Compounds of the benzoquinone class are exemplified by geldanamycin (including its synthetic derivatives 17-allylamino-17-demethoxygeldanamycin (17-AAG), 17-N,N-dimethylaminoethylamino-17-demethoxygeldanamycin (DMAG), dihydrogeldanamycin and herbamycin. The benzohydroquinone class is exemplified by macbecin.

The terms “dispersion,” “colloid,” and “emulsion” have meanings in the art consistent with Remington, THE SCIENCE AND PRACTICE OF PHARMACY, 20th Edition (2000) and denote multiphasic systems comprised of two or more ingredients that are not completely miscible in one another. Dispersions may be classified into different groups based on the size of the dispersed particles. Colloidal dispersions are characterized by dispersed particles in the range of approximately 1 nm to 0.5 μm. Coarse dispersions are characterized by particle sizes exceeding 0.5 μm, and include suspensions and emulsions. For the most part, the different types of dispersions can be detected by light-scattering and/or microscopic techniques, including, e.g. electron microscopy.

An “emulsion” is a dispersed system containing at least two immiscible liquid phases—usually water and oil accompanied by a surfactant/emulsifying agent—and having particles ranging in size of from about 0.1 μm to about 100 μm.

The term “oral bioavailability” refers to an estimate of the amount of active pharmaceutical ingredient that is systemically available in the serum after oral administration. The apparent absolute oral bioavailability (F) is estimated from the ratio of the area under a plasma concentration vs. time after oral administration curve to a similar curve after intravenous injection, both normalized to the dosage, using the following equation:

% F=[(Oral AUC₍₀₋₆ₕ₎×I.V. Dose)/(I.V. AUC₍₀₋₆ₕ₎×Oral Dose)]*100.

A “physiologically acceptable carrier” refers to a carrier or diluent that does not cause significant irritation to an organism and does not abrogate the biological activity and properties of the administered compound. Depending on the formulation, the diluents can be a solid such as calcium carbonate, sodium carbonate, lactose, calcium phosphate or sodium phosphate, or a liquid, such as water or oils.

An “excipient” refers to a non-toxic pharmaceutically acceptable substance added to a pharmacological composition to facilitate the processing, administration and pharmaceutics properties of a compound. Excipients may include but are not limited to fillers or diluents, glidants, lubricants, disintegrants, binders, solubilizers, stabilizers/bulking agents, and various functional and non-functional coatings.

“Fillers and diluents” are inert agents which are added to increase the bulk in order to make a tablet a practical size for compression. Examples of fillers or diluents are, but are not limited to, calcium sulfate, calcium carbonate, sodium carbonate, dicalcium phosphate, sodium phosphate, lactose, mannitol, microcrystalline cellulose, and Neusilin, polyethyleneglycol, water, and various oils, such as peanut oil, liquid paraffin, and olive oil.

“Glidants and lubricants” are agents which improve the rate of flow of the tablet granulation, prevent adhesion of the tablet material to the surface of the dies and punches, reduce interparticle friction and facilitate the ejection of the tablets from the die cavity. Commonly used lubricants include talc, magnesium stearate, calcium stearate, stearic acid and hydrogenated vegetable oils. Examples of glidants are, but are not limited to, colloidal silicon dioxide and talc.

“Disintegrants” are substances that are added to a tablet to facilitate its breakup or disintegration after administration. Examples of disintegrants are, but are not limited to, sodium crosscarmellose, crospovidone, microcrystalline cellulose, corn starch, alginic acid, and sodium starch glycolate.

“Binders and granulators” are agents which impart cohesive qualities to the powdered drug. The impart a cohesiveness to the tablet formulation which insures the tablet remains intact after compression, as well as improving free-flowing qualities by the formulation of granules od desired hardness and size. Examples of binders are, but are not limited to, starch, gelatin, polyvinylpyrrolidone, polyethylene oxide, polyethylene glycol, hydroxymethylpropyl cellulose, carboxymethylcellulose sodium.

The term “solubilizers” are agents in which the active pharmaceutical ingredient has intrinsic solubility or an agent that enhances the dissolution rate and/or overall aqueous solubility of the active pharmaceutical ingredient. Exemplary solubilizers for the ansamycins include, but are not limited to, phospholipids, polyethylene glycols of various molecular weights, sodium lauryl sulfate, Tween 80 or other synthetic surfactants, Solutol HS15, Labrasol, propylene carbonate, Transcutol HP and glycofurol, oleic acid and short-, medium- or ling-chain triglycerides. Applicants expressly exclude dimethylsulfoxide (DMSO) as a solubilizer for the formulations of the invention.

The term “surfactants” or “wetting agents” are agents which delay a non-homogeneous and thermodynamically unstable dispersion to separate into the minimum possible surface area of contact between phases. Surfactants operate by accumulating at the phasic interface and providing an energy barrier to aggregation and coalescence. Surfactants which may be used with the invention include phospholipids, anionic surfactants, cationic surfactants and non-ionic surfactants. The surfactant of the invention is typically present in concentrations of about 0.5-75% w/w based on the amount of the water and/or other components into which the surfactant is dissolved. Preferably, the surfactant is present in a concentration of about 0.5-50% w/w.

Examples of anionic surfactants include sodium lauryl sulfate, lauryl sulfate triethanolamine, sodium polyoxyethylene lauryl ether sulfate, sodium polyoxyethylene nonylphenyl ether sulfate, polyoxyethylene lauryl ether sulfate triethanolamine, sodium cocoylsarcosine, sodium N-cocoylmethyltaurine, sodium polyoxyethylene (coconut)alkyl ether sulfate, sodium diether hexylsulfosuccinate, sodium α-olefin sulfonate, sodium lauryl phosphate, sodium polyoxyethylene lauryl ether phosphate and perfluoroalkyl carboxylate salt (manufactured by Daikin Industries Ltd. under the trade name of UNIDINE DS-101 and 102).

Examples of cationic surfactants include dialkyl(C₁₂-C₂₂)dimethylammonium chloride, alkyl(coconut)dimethylbenzylammonium chloride, octadecylamine acetate salt, tetradecylamine acetate salt, tallow alkylpropylenediamine acetate salt, octadecyltrimethylammonium chloride, alkyl(tallow)trimethylammonium chloride, dodecyltrimethylammonium chloride, alkyl(coconut)trimethylammonium chloride, hexadecyltrimethylammonium chloride, biphenyltrimethylammonium chloride, alkyl(tallow)-imidazoline quaternary salt, tetradecylmethylbenzylammonium chloride, octadecyidimethylbenzylammonium chloride, dioleyidimethylammonium chloride, polyoxyethylene dodecylmonomethylammonium chloride, polyoxyethylene alkyl(C₁₂-C₂₂)benzylammonium chloride, polyoxyethylene laurylmonomethyl ammonium chloride, 1-hydroxyethyl-2-alkyl(tallow)-imidazoline quaternary salt, a silicone cationic surfactant having a siloxane group as a hydrophobic group, and a fluorine-containing cationic surfactant having a fluoroalkyl group as a hydrophobic group (manufactured by Daikin Industries Ltd. under the trade name of UNIDINE DS-202).

Examples of nonionic surfactants include polyoxyethylene lauryl ether, polyoxyethylene tridecyl ether, polyoxyethylene cetyl ether, polyoxyethylene polyoxypropylene cetyl ether, polyoxyethylene stearyl ether, polyoxyethylene oleyl ether, polyoxyethylene nonylphenyl ether, polyoxyethylene octylphenyl ether, polyoxyethylene monolaurate, polyoxyethylene monostearate, polyoxyethylene monooleate, sorbitan monolaurate, sorbitan monostearate, sorbitan monopalmitate, sorbitan monostearate, sorbitan monooleate, sorbitan sesquioleate, sorbitan trioleate, polyoxyethylene sorbitan monolaurate, polyoxyethylene sorbitan monopalmitate, polyoxyethylene sorbitan monostearate, polyoxyethylene sorbitan monooleate, polyoxyethylene polyoxypropylene block polymer, polyglycerin fatty acid ester, polyether-modified silicone oil (manufactured by Toray Dow Corning Silicone Co., Ltd. under the trade names of SH3746, SH3748, SH3749 and SH3771), perfluoroalkyl ethyleneoxide adduct (manufactured by Daikin Industries Ltd. under the trade names of UNIDINE DS-401 and DS-403), fluoroalkyl ethyleneoxide adduct (manufactured by Daikin Industries Ltd. under the trade name of UNIDINE DS-406), and perfluoroalkyl oligomer (manufactured by Daikin Industries Ltd. under the trade name of UNIDINE DS-451).

The term “phospholipid” includes any lipid containing phosphoric acid as mono- or di-ester. The phospholipids of the invention may be synthetic, natural, or semi-synthetic and preferably, although not necessarily, share identity with known cellular membrane phospholipids such as phosphoglycerides and sphingomyelin.

Phosphoglycerides are derived from glycerol, a three-carbon alcohol, and possess a glycerol backbone esterified to two fatty acid chains via two glycerol hydroxyl groups, and esterified to phosphoric acid via the remaining hydroxyl group to form an intermediate, phosphatidate. The fatty acid chains typically contain between 14 and 24 carbon atoms, with 16 and 18 being the most common. The chains may be either saturated or unsaturated. The phosphate group itself is then esterified to the hydroxyl group of one of several different alcohols, with the most common being serine, ethanolamine, choline, glycerol, and inositol. Exemplary phosphoglycerides include, but are not limited to, phosphatidylcholine (PC), phosphatidylserine (PS), phosphatidylinositol (PI), phosphatidylethanolamine (PE). Sphingomyelin is derived from sphingosine, an amino alcohol, which contains a long, unsaturated hydrocarbon chain. In sphingomyelin, the amino group of the sphingosine backbone is linked to a fatty acid by an amide bond. In addition, the primary hydroxyl group of sphingosine is esterified to phosphoryl choline. See, e.g., Stryer, BIOCHEMISTRY, Second Edition, pp. 206-211 (1981).

Additionally, phosphoglycerides also include lecithins. “Lecithins” are naturally occurring mixtures of diglycerides of stearic, palmitic, and oleic acids, linked to the choline ester of phosphoric acid. Preferred phospholipids for use with the invention are soya lecithin, e.g., Phospholipon 900 as supplied by American Lecithen Company (Oxford, Conn., USA). Other commercial sources and methods of preparation are known to the skilled artisan.

Most phospholipids are amphoteric surfactants, because of their polar zwitterionic head group and the hydrocarbon tails. These comprise the majority of the lipid component of cell membranes in most living organisms, including humans. A group of phospholipids that is not amphoteric includes, e.g., phosphatidylserine and phosphatidylinositol. Yet another includes the cephalins.

The phospholipid surfactants of the invention are typically present in concentrations of about 5-75% w/w based on the amount of the water and/or other components into which the surfactant is dissolved. Preferably, the phospholipid is present in a concentration of about 10-50% w/w, most preferably about 10-25% w/w. To prevent or minimize oxidative degradation or lipid peroxidation, antioxidants, e.g., alpha-tocopherol and butylated hydroxytoluene, may be included in addition to, or as an alternative to, oxygen deprivation (e.g., formulation in the presence of inert gases such as nitrogen and argon, and/or the use of light resistant containers).

“Stabilizer/bulking agents” are a type of excipient that generally provides mechanical support for a lyophile formulation by allowing the dry formulation matrix to maintain its conformation. Preferred are sugars. Sugars as used herein include but are not limited to monosaccharides, disaccharides, oligosaccharides and polysaccharides. Specific examples include but are not limited to fructose, glucose, mannose, trehalose, sorbose, xylose, maltose, lactose, sucrose, dextrose, and dextran. Sugar also includes sugar alcohols, such as mannitol, sorbitol, inositol, dulcitol, xylitol and arabitol. Mixtures of sugars may also be used in accordance with this invention. Various bulking agents, e.g., glycerol, sugars, sugar alcohols, and mono and disaccharides may also serve the function of isotonizing agents.

Bulking agents for use with the invention are limited only by chemico-physical considerations, such as solubility, ability to preserve the droplet size and emulsion integrity during freezing, drying, storage and rehydration and lack of reactivity with the active drug/compound, and limited as well by the form, i.e., solid or liquid, of the formulation. It is preferred that the bulking agents be chemically inert to drug(s) and have no or extremely limited detrimental side effects or toxicity under the conditions of use.

A preferred bulking agent for the invention is sucrose. Other bulking agents which may be substituted include but are not limited to polyvinylpyrrolidone (PVP) and mannitol.

The term “lyophilization” means the removal or substantial removal of liquid from a sample by sublimation. Solvent/aqueous phase removal may be accomplished using any procedure but is preferably accomplished under reduced pressure, i.e., vacuum, at any reasonable temperature and pressure, including at room temperature with a stream of nitrogen, as long as suitable to preserve the functional integrity of the pharmaceutical agent. Commercially available rotary evaporation devices exist to accomplish solvent removal. Other devices and methods are known to the skilled artisan.

The terms “lyophilization”, “lyophilizing” and “lyophilized” do not necessarily imply 100% elimination of solvent and solution, and may entail lesser percentages of removal. Substantial removal is preferred, preferably about 95% removal.

The term “hydrating” or “rehydrating” means adding an aqueous solution, e.g., water or a physiologically compatible buffer such as Hanks's solution, Ringer's solution, physiological saline buffer, or 5% dextrose in water.

The term “about” means including and exceeding up to 15% the specific endpoint(s) designated. Thus the range is broadened.

The term “optionally” denotes that the step or component following the term may but need not be a part of the method or formulation.

### II. Preparation of Oral Formulations

A. Preparation of Ansamycins

Ansamycins according to this invention may be synthetic, naturally-occurring, or a combination of the two, i.e., “semi-synthetic,” and may include dimers and conjugated variant and prodrug forms. Some exemplary benzoquinone ansamycins useful in the various embodiments of the invention and their methods of preparation include but are not limited to those described, e.g., in U.S. Pat. No. 3,595,955 (describing the preparation of geldanamycin), No. 4,261,989, No. 5,387,584, and No. 5,932,566 and those described in the “EXAMPLE” section (Examples 1-12), below. Geldanamycin is also commercially available, e.g., from CN Biosciences, an Affiliate of Merck KGaA, Darmstadt, Germany, headquartered in San Diego, Calif., USA (cat. no. 345805). 17-N,N-dimethylaminoethylamino-17-desmethoxy-geldanamycin (DMAG) is commercially available from EMD/Calbiochem. The biochemical purification of the geldanamycin derivative, 4,5-dihydrogeldanamycin and its hydroquinone from cultures of Streptomyces hygroscopicus (ATCC 55256) are described in International Application Number PCT/US92/10189, assigned to Pfizer Inc., published as WO 93/14215 on Jul. 22, 1993, and listing Cullen et al. as inventors; an alternative method of synthesis for 4,5-dihydrogeldanamycin by catalytic hydrogenation of geldanamycin is also known. See e.g., “Progress in the Chemistry of Organic Natural Products,” Chemistry of the Ansamycin Antibiotics, 1976 33:278. Other ansamycins that can be used in connection with various embodiments of the invention are described in the literature cited in the “Background” section and also in the “Summary” section, above.

17-AAG may be prepared from geldanamycin by reacting with allyamine in dry THF under a nitrogen atmosphere. The crude product may be purified by slurrying in H₂O:EtOH (90:10), and the washed crystals obtained have a melting point of 206-212° C. by capillary melting point technique. A second product of 17-AAG can be obtained by dissolving and recrystallizing the crude product from 2-propyl alcohol (isopropanol). This second 17-AAG product has a melting point between 147-153° C. by capillary melting point technique. The two 17-AAG products are designated as the low melt form and high melt form. The stability of the low melt form may be tested by slurring the crystals in the solvent (H₂O:EtOH (90:10)) from which the high melt form was purified; no conversion to the high melt form was observed. See Examples 1-2 for details of the preparation of the two polymorphic forms of 17-AAG.

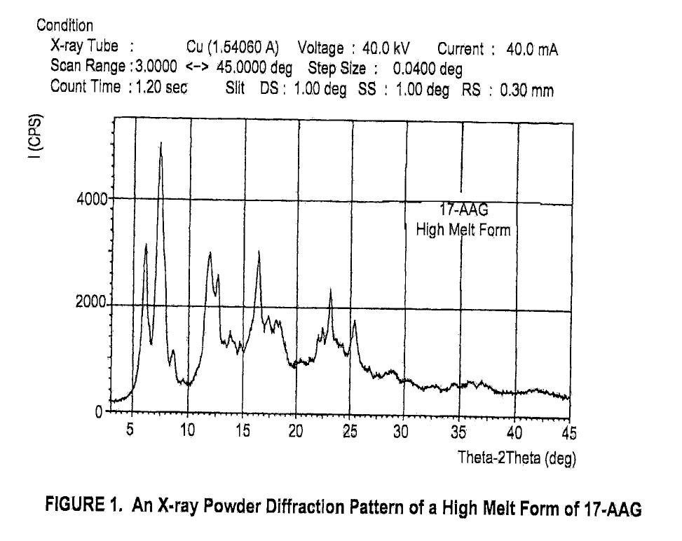

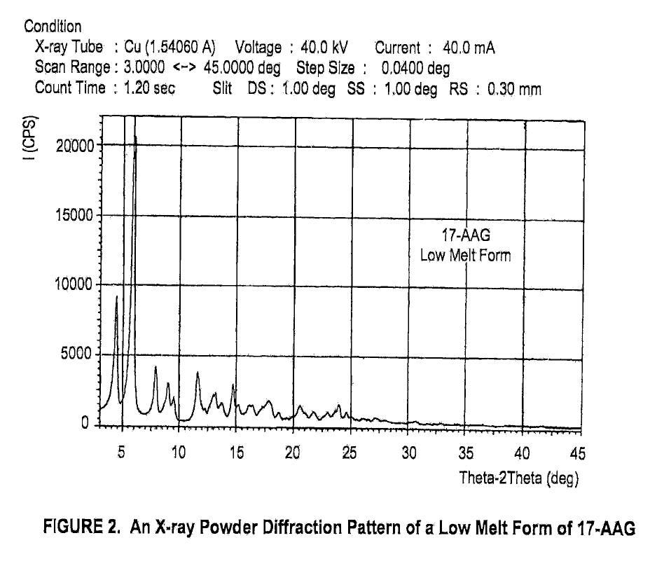

The distinctiveness of the polymorphic forms may be assessed by X-ray powder diffraction and by deferential scanning calorimetry. Distinctively different X-ray powder diffraction patterns/spectra generally indicate that the materials have different crystalline forms. FIG. 1 shows the X-ray powder diffraction pattern of the high melt form which include three peaks at 7.40 degree, 6.08 degree and 11.84 degree two-theta angles. FIG. 2 shows the X-ray powder diffraction pattern of the low melt form of 17-AAG which include three peaks at 5.85 degree, 4.35 degree and 7.90 degree two-theta angles. Accordingly, the X-ray powder diffraction spectra of the two polymorphic forms were distinctly different, thus confirming that the high melt and low melt 17-AAG contain distinct polymorphic forms of 17-AAG.

The peak locations and intensities of the X-ray powder diffraction patterns for the high melt form and low melt form of 17-AAG are summarized in Table 1 and Table 2, respectively.

TABLE 1
X-Ray Powder Diffraction Pattern of A High Melt 17-AAG
17-AAG High Melt Form

| | | | | | | | |
|---|---|---|---|---|---|---|---|
| | | | | | | | Integrated |
| | peak | 2Theta | d | | FWHM | Intensity | Int |
| no. | no. | (deg) | (A) | I/I1 | (deg) | (Counts) | (Counts) |

# Strongest 3 peaks

| | | | | | | | |
|---|---|---|---|---|---|---|---|
| 1 | 2 | 7.4042 | 11.92989 | 100 | 0.88940 | 3462 | 77678 |
| 2 | 1 | 6.0824 | 14.51916 | 57 | 0.73690 | 1964 | 40942 |
| 3 | 5 | 11.8400 | 7.46851 | 52 | 0.81900 | 1810 | 32565 |

# Peak Data List

| | | | | | | |
|---|---|---|---|---|---|---|
| 1 | 6.0824 | 14.51916 | 57 | 0.73690 | 1964 | 40942 |
| 2 | 7.4042 | 11.92989 | 100 | 0.88940 | 3462 | 77678 |
| 3 | 8.6000 | 10.27358 | 14 | 0.63020 | 472 | 9907 |
| 4 | 10.7200 | 8.24615 | 4 | 0.34660 | 125 | 1866 |
| 5 | 11.8400 | 7.46851 | 52 | 0.81900 | 1810 | 32565 |
| 6 | 12.4800 | 7.08691 | 40 | 0.91960 | 1386 | 36608 |
| 7 | 13.8800 | 6.37508 | 16 | 0.00000 | 546 | 0 |
| 8 | 14.7200 | 6.01312 | 11 | 0.00000 | 366 | 0 |
| 9 | 16.3120 | 5.42966 | 45 | 0.88790 | 1566 | 50640 |
| 10 | 17.3200 | 5.11587 | 22 | 0.00000 | 746 | 0 |
| 11 | 18.1600 | 4.88108 | 21 | 1.36660 | 711 | 26508 |
| 12 | 20.4400 | 4.34147 | 3 | 1.38660 | 110 | 4924 |
| 13 | 22.2400 | 3.99400 | 15 | 0.93120 | 524 | 11702 |
| 14 | 23.1340 | 3.84163 | 28 | 0.82570 | 961 | 22215 |
| 15 | 24.1200 | 3.68678 | 12 | 0.00000 | 400 | 0 |
| 16 | 25.3229 | 3.51431 | 21 | 0.86220 | 717 | 20392 |
| 17 | 26.6400 | 3.34347 | 3 | 0.66660 | 116 | 3106 |
| 18 | 28.7575 | 3.10191 | 4 | 1.19500 | 153 | 6842 |
| 19 | 36.0400 | 2.49007 | 4 | 1.77600 | 143 | 7021 |
| 20 | 36.9200 | 2.43271 | 4 | 1.60000 | 130 | 4256 |

TABLE 2
X-Ray Powder Diffraction Pattern of A Low Melt 17-AAG
17-AAG Low Melt Form

| | | | | | | | |
|---|---|---|---|---|---|---|---|
| | | | | | | | Integrated |
| | peak | 2Theta | d | | FWHM | Intensity | Int |
| no. | no. | (deg) | (A) | I/I1 | (deg) | (Counts) | (Counts) |

# Strongest 3 peaks

| | | | | | | | |
|---|---|---|---|---|---|---|---|
| 1 | 2 | 5.8457 | 15.10652 | 100 | 0.40550 | 14036 | 168505 |
| 2 | 1 | 4.3495 | 20.29913 | 44 | 0.33410 | 6212 | 68273 |
| 3 | 3 | 7.9044 | 11.17604 | 20 | 0.33160 | 2744 | 26991 |

# Peak Data List

| | | | | | | |
|---|---|---|---|---|---|---|
| 1 | 4.3495 | 20.29913 | 44 | 0.33410 | 6212 | 68273 |
| 2 | 5.8457 | 15.10652 | 100 | 0.40550 | 14036 | 168505 |
| 3 | 7.9044 | 11.17604 | 20 | 0.33160 | 2744 | 26991 |
| 4 | 8.6400 | 10.22611 | 5 | 0.36300 | 709 | 6793 |
| 5 | 8.9975 | 9.82058 | 14 | 0.39580 | 1958 | 16858 |
| 6 | 9.5200 | 9.28272 | 8 | 0.27580 | 1159 | 10258 |
| 7 | 11.6397 | 7.59657 | 18 | 0.39840 | 2557 | 26916 |
| 8 | 12.2000 | 7.24892 | 3 | 0.37220 | 482 | 6182 |
| 9 | 12.6800 | 6.97557 | 5 | 0.35260 | 662 | 6166 |
| 10 | 13.1200 | 6.74261 | 9 | 0.44280 | 1264 | 13808 |
| 11 | 13.7200 | 6.44906 | 5 | 0.40080 | 701 | 8364 |
| 12 | 14.6978 | 6.02215 | 12 | 0.32910 | 1621 | 13247 |
| 13 | 15.1600 | 5.83957 | 4 | 0.35560 | 562 | 5980 |
| 14 | 16.1200 | 5.49390 | 4 | 0.44100 | 564 | 5716 |
| 15 | 16.4000 | 5.40073 | 4 | 0.33740 | 579 | 4433 |
| 16 | 17.6523 | 5.02031 | 6 | 0.94470 | 882 | 21567 |
| 17 | 20.5468 | 4.31915 | 5 | 0.54150 | 714 | 16199 |
| 18 | 23.5200 | 3.77945 | 3 | 0.32680 | 428 | 8157 |
| 19 | 23.8800 | 3.72328 | 6 | 0.32180 | 841 | 10452 |

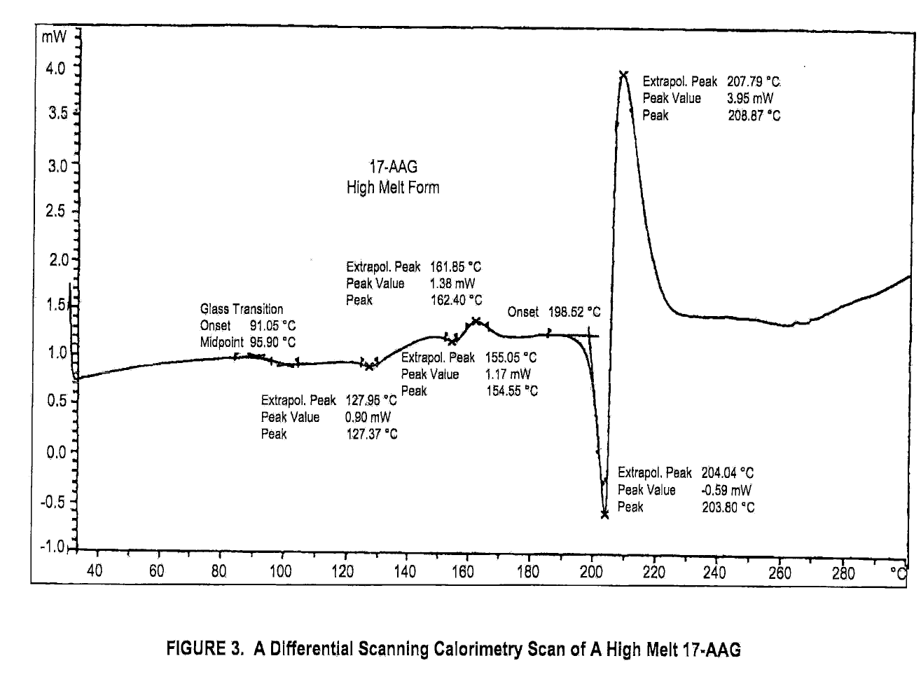

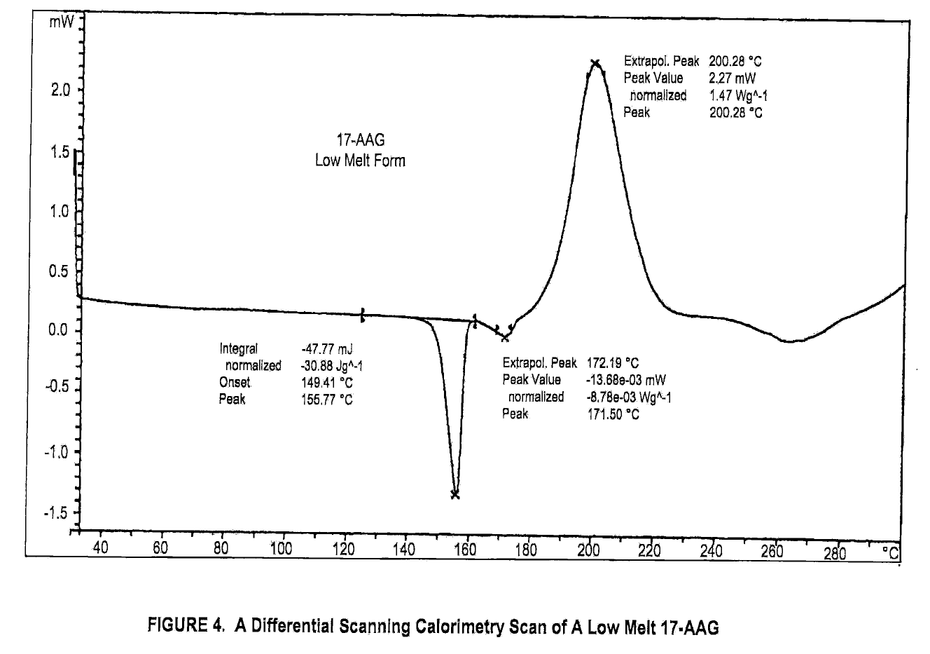

The distinction of the high melt and low melt form is also confirmed by differential scanning calorimetry (DSC). FIG. 3 shows the DSC scan of a high melt form of 17-AAG showing a single endotherm at 204° C. FIG. 4 shows the DSC scan of a low melt form of 17-AAG showing two endotherms centered at 156° C. and at 172° C., and the 172° C. endotherm ends at about 176° C.

The presence of two endotherms in the DSC trace of the low melt form of 17-AAG is indicative of the presence of at least two polymorphic forms. Various mixtures of the polymorphic forms of 17-AAG having DSC temperature below 176° C. is generally referred to as the low melt form of 17-AAG or low melt 17-AAG. Specific mixtures will be referred to by their characteristic DSC temperatures, e.g., the low melt form of 17-AAG having a melting temperature of 156° C. In addition, it is well known that ansamycins exist in an amorphous form.

The formulations of the invention contemplate all the various polymorphic forms of the ansamycins, particularly, all the polymorphic forms of 17-AAG in a polymorphic mixture or an individual polymorph, or amorphous form.

The thermal analysis data (i.e., DSC and TGA) are summarized in TABLE 3 below. It is noted that there is a difference of a few degrees of the melting temperatures of the polymorphs reported in Examples 1-3 when compared to the melting temperature measured by DSC. This difference can be attributed to the analytical technique used. The melt temperature in Examples 1-3 were measured by a standard capillary technique. With the very dark colored crystals of 17-AAG, it is difficult to precisely determine the onset and completion of the melt cycle.

TABLE 3
Thermal Analysis of High Melt vs Low Melt 17-AAG.

| Test | Low Melt Form | High Melt Form |
|---|---|---|
| TGA | No Weight Loss Observed | 3.5% Weight Loss |
| DSC (peak) melt | 156° C. and 172° C. | 204° C. |

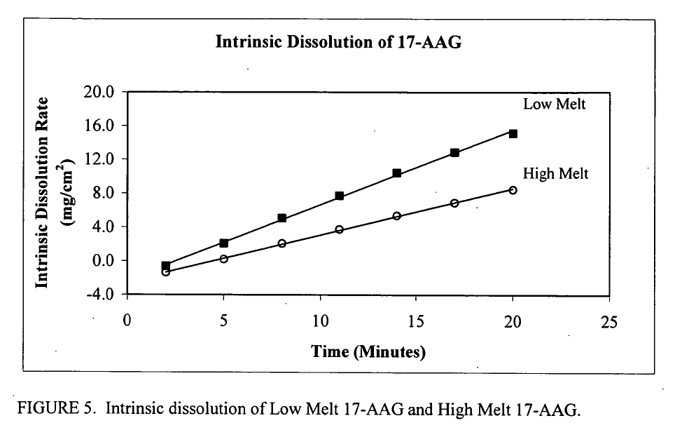

The performance of an active pharmaceutical ingredient (i.e., dissolution rate) can be affected by polymorphic state. The intrinsic dissolution of the high melt and low melt forms of 17-AAG were determined in ethanol. Ethanol was chosen as the solvent since 17-AAG is soluble in ethanol. The low melt form of 17-AAG had a 60% higher intrinsic dissolution rate (0.885 mg/cm²/min) than the high melt form (0.550 mg/cm²/min) as shown in FIG. 5.

The rapid dissolution rate of the low melt form may provide improved bio-availability as drug is absorbed from solution in the GI tract, the low melt form can rapidly dissolve and maintain a saturated solubility keeping drug in solution and available for absorption.

B. Preparation of the Oral Dosage Form

The oral formulations of the invention may be in the form of a capsule, tablet, liquid filled/semi-solid capsule, and an oral solution. The critical performance characteristic of the dosage form is to provide for an immediate release and rapid dissolution of the drug, e.g., 17-AAG, from the dosage in GI fluids.

Compositions intended for oral use may be prepared according to any method known to the art for the manufacture of pharmaceutical compositions, and such compositions may contain, in addition to the general excipients, one or more agents selected from the group consisting of sweetening agents, flavoring agents, coloring agents and preserving agents in order to provide pharmaceutically elegant and palatable preparations.

The ansamycins may be first formulated into a liquid and then process into various oral forms.

1. Preparation of Oral Liquid Formulations

For example, 17-AAG can be formulated as an oral solution containing solubilizing/complexing agents (e.g., phospholipids, sodium lauryl sulfate, Tween 80, polyethylene glycols of various molecular weights, polyethylene glycols of various molecular weights, ethanol, Solutol HS15, Labrasol, propylene carbonate, Transcutol HP, glycofural, oleic acid and short-, medium- or long-chain triglycerides, synthetic and non-synthetic surfactants and combinations thereof) at concentrations up to 50 mg/mL. The oral solution can be provided in ready to use fashion, or as a pre-packaged powder blend containing the drug and necessary excipients (e.g., phospholipids, sodium lauryl sulfate) that may be combined or mixed with water at the time of use. The oral powder can contain flavoring agents such as sucrose, mannitol, or grape flavor agents to make the product more palatable.

Elixir formulations of 17-AAG are possible containing various solvents such as glycerol, Labrafil, Labrasol, polyethylene glycols of various molecular weights, Tween 80, Tween 20, and Solutol HS15, propylene carbonate, ethanol, Transcutol HP, glycofurol, oleic acid, short-, medium- or long-chain triglycerides, and combinations thereof, and flavoring agents (i.e., tinctures such as orange, strawberry, etc.) combined with various sugars (e.g., sucrose, mannitol, xylitol. Sucrolose, sorbitol, various syrups) or other sweetening agents such as saccharin or aspartame. Such formulations may also contain demulcents, preservatives, coloring agents and antioxidants. The elixir formulation can contain up to 20 mg/mL of 17-AAG.

17-AAG can be formulated as an oral suspension. The suspension formulation can be prepared using size reduced 17-AAG (down to 1 μm) in the preparation of the oral product in admixture with excipients suitable for the manufacture of aqueous suspensions. Such excipients are suspending agents, for example microcrystalline cellulose, sodium carboxymethylcellulose, methylcellulose, hydroxypropylmethyl-cellulose, sodium alginate, polyvinyl-pyrrolidone, gum tragacanth and gum acacia, xanthan gum, etc. The suspension can contain polyethylene glycol and/or other pharmaceutically acceptable solvents for oral use. Exemplary solvents, include but are not limited to, glycerol, Labrafil, Labrasol, polyethylene glycols of various molecular weights, Tween 80, Tween 20, and Solutol HS15, propylene carbonate, ethanol, Transcutol HP, glycofurol, oleic acid, short-, medium- or long-chain triglycerides, and combinations thereof. The solubility of 17-AAG in selected solvents are listed in TABLE 4. The suspension may further include dispersing or wetting agents such as sodium lauryl sulfate (SLS), Tween 80, a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

pH can be manipulated in these liquid formulations of the invention using suitable acids and bases, e.g., hydrochloric acid and sodium hydroxide for adjusting sodium acetate buffered compositions. In addition to or as an alternative to the use of sodium acetate, other buffers can be used, e.g., histidine (not more than 5 mM; pH 5), lactic acid (˜10-20 mM; pH ˜4), valine (˜10-50 mM; pH 3), etc.

Dispersion and particle size reduction of a suspension can be effected by a variety of well known techniques, e.g., mechanical mixing, homogenization (e.g., using a polytron or Gaulin high-energy-type instrument), vortexing, and sonication. Sonication can be effected using a bath-type or probe-type instrument. Microfluidizers are commercially available, e.g., from Microfluidics Corp., Newton, Mass., are further described in U.S. Pat. No. 4,533,254, and make use of pressure-assisted passage across narrow orifices, e.g., as contained in various commercially available polycarbonate membranes. Low pressure devices also exist that can be used. These high and low pressure devices can be used to select for and/or modulate particle size.

Sterilization of a liquid dispersion can be achieved by various filtration techniques. Filtration can include a pre-filtration through a larger diameter filter, e.g., a 0.45 micron filter, and then through a smaller filter, e.g., a 0.2 micron filter. A preferred filter medium is cellulose acetate (Sartorius-Sartobran™). Alternatively, the formulation may be directly extruded through a 0.2 micron or smaller filter. In any event, extrusion through a microchannel filter of 0.2 micron or smaller pore size effectively filter-sterilizes, making additional filter-sterilization unnecessary.

a. Phospholipid Formulation

In one embodiment, the active pharmaceutical ingredient, 17-AAG, is formulated as a 1% (w/w) aqueous phospholipid dispersion. The formulation is prepared by simply mixing 17-AAG an aqueous dispersion of phospholipids in a high shear mixture for a short duration and then slowly stirring to remove entrained air. Any phospholipids previously described, such as Phospholipon 90G, phosphatidylcholine, phosphatidylserine, phosphatidylinositol, phosphatidyl ethanolamine, may be used. During the formulation process, other excipients such as buffers, tonicity adjustment agents, and process aids may be added. Such a formulation composition is below:

| Ingredient | % by weight | Function |
|---|---|---|
| 17-AAG | 1.0 | Active Ingredient |
| L-histidine | 0.1 | Buffer |
| Sucrose | 7.5 | tonicity/cryoprotectant |
| Phospholipid | 18.8 | lipid complex/carrier |
| Ethanol | 0.5 | process aid |
| Tween 80 | 0.5 | process aid |
| Water | 71.5 | Diluent |

The 17-AAG dispersion may be microfluidized at 16-19,000 psi to reduce the particle size of the dispersion from about 5 μm to 0.1-0.5 μm (mean particle size). Microfluidization can be accomplished using a Model 11OS microfluidizer (Microfluidics Inc., Newton, Mass., USA).

The dispersions can be filter-sterilized using a sterile 0.2 micron Sartorius Sartobran P capsule filter (500 cm²) (Sartorius A G, Goettingen, Germany), with pressure up to 60 psi used to maintain a smooth and continuous flow. The filtrate can be immediately processed into other oral formulations such as oral solutions, tablets or capsules using standard techniques which are known in the art. The filtrate can also be collected, froze, or lyophilized for future use.

Lyophilization results in a product that is relatively stable and convenient for storage, shipping, and handling. Upon hydration and adjustment to a suitable concentration, administration may be conveniently made to a patient. Condition for lyophilization can be found in EXAMPLE 15.

In other embodiments, pharmaceutical acceptable co-solvents may be added to the phospholipid formulation to further enhance the solubility of the ansamycins. Many suitable co-solvents which are known in the art may be used. Exemplary solvents includes, but are not limited to, those listed in TABLE 4 below; the 17-AAG solubility in these solvents are also listed. The resulted formulation can then be combined with any combinations of the general excipients: fillers, glidants, lubricants, disintegrants, binders, solubilizers to form the various oral compositions.

TABLE 4
Solubility of 17-AAG in Organic Solvents
Suitable for Oral Adminstration

| | Solubility |
|---|---|
| Solvent | (mg/mL) |
| Glycerol | 1.1 |
| Labrafil† | 2.4 |
| Labrasol▾ | 16.1 |
| Polyethylene glycol 400 | 12.9 |
| Tween 80 | 33.8 |
| Solutol HS15 | 12.4 |
| Propylene Carbonate | 12.8 |
| Transcutol HP* | 57.7 |
| Glycofurol | 47.0 |

†Apricot Kernol Oil PEG-6 Esters
▾PEG-8 Caprylic/Capric Glycerides
*Ethoxydiglycol

b. Non-lipid PEG-Tween Oral Formulation of 17-AAG

A PEG-Tween oral formulation can be prepared by first mixing polyethylene glycol with Tween 80, e.g. in a ratio of 4:1 w/w to form a viscous solution. Polyethylene glycol of various molecular weight may be used. In one embodiment PEG400 is used. 17-AAG, can then be added and the solution stirred for about one hour with intermittent sonication to dissolve the 17-AAG. Varing amounts of 17-AAG may be added. In one embodiment, 17-AAG of about 1% w/w was used. The resulting solution is a deep purple viscous solution.

c. Non-lipid Sodium Lauryl Sulfate (SLS) Formulation of 17-AAG

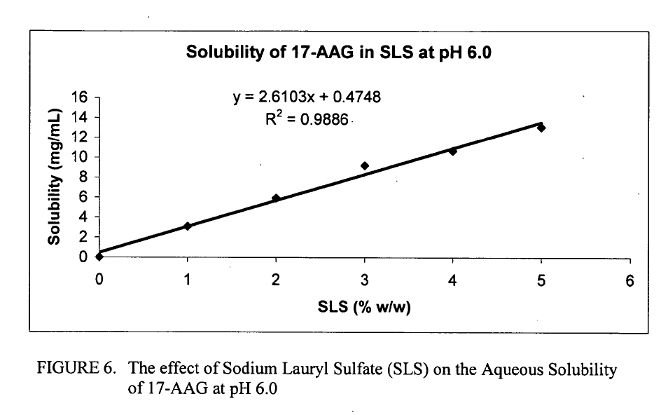

Applicants have observed that sodium lauryl sulfate is effective in solubilizing 17-AAG in an aqueous medium. FIG. 6 shows that aqueous solubility increases linearly with increasing concentration of SLS. Formulations of the invention may contain as much as 50% SLS.

The invention contemplates using SLS alone or in combination with other excipients, especially with other co-solvents or solubilizers. Examples of such co-solvents include, but are not limited to, glycerol, Labrafil, Labrasol, polyethylene glycols of various molecular weights, Tween 80, Tween 20, and Solutol HS15, propylene carbonate, ethanol, Transcutol HP, glycofurol, oleic acid, short-, medium- or long-chain triglycerides, and combinations thereof.

The invention also contemplates formulations which include combinations of phospholipids and SLS. The invention further contemplates formulations which include combination of SLS with other solubilizers and co-solvents, such as ethanol, PEG, Tween 80, Solutol HS15, Labrtasol, propylene carbonate, Transcutol, glycofurol, oleic acid and short-, medium-, or long-chain triglycerides, other synthetic and non-synthetic surfactant, and combinations thereof.

2. Preparation of Solid/Semi-Solid Oral Formulations

In one embodiment of the invention, 17-AAG is formulated into tablet for oral delivery. Methods of compounding solid and semi-solid tablets and capsules are known to those skilled in the art and may also be found in REMINGTON'S PHARMACEUTICAL SCIENCES, Osol, A. Ed.; 18th Edition, Mack, 1990.

A tablet formulation of 17-AAG can be composed of excipients that are suitable for the manufacture of tablets which include, but are not limited to: fillers, glidants, lubricants, disintegrants, binders, solubilizers, and various functional and non-functional coatings, which are generally known in the art. Examples of fillers are, but are not limited to, lactose, mannitol, microcrystalline cellulose, and Neusilin. Examples of glidants are, but are not limited to, colloidal silicon dioxide and talc. Examples of disintegrants are, but are not limited to, sodium crosscarmellose, crospovidone, and sodium starch glycolate. Examples of binders are, but are not limited to, polyvinylpyrolidone, polyethylene oxide, polyethylene glycols, hydroxymethylpropyl cellulose, and carboxymethylcellulose sodium. Examples of solubilizers are, but are not limited to, phospholipids, polyethylene glycols of various molecular weights, ethanol, sodium lauryl sulfate, Tween 80, Solutol HS15, Labrasol, propylene carbonate, Transcutol HP, glycofural, oleic acid and short-, medium- or long-chain triglycerides, and combinations thereof.

The tablet formulation can be prepared by a number of processes such as, but not limited to, direct compression, dry granulation, wet granulation, fluid bed granulation, and various common techniques involving spray dry technologies. The formulated powder of 17-AAG can be filled into gelatin capsules or compressed into a tablet. A tablet formulation may be coated for aesthetic and/or taste masking purposes.

In another embodiment of the invention, the active pharmaceutical ingredient may be first solubilized in an organic solvent such as of glycerol, Labrafil, Labrasol, polyethylene glycols of various molecular weights, Tween 80, Tween 20, and Solutol HS15, propylene carbonate, ethanol, Transcutol HP, glycofurol, oleic acid, short-, medium- or long-chain triglycerides, and combinations thereof. The solubility of 17-AAG in selected solvents are listed in TABLE 4. The solvents may be used alone or in combination with other solvents and/or excipients such as sodium lauryl sulfate. The resulted solution or slurry is then combined with any combination of the general excipients: fillers, glidants, lubricants, disintegrants, binders, solubilizers, etc., to form a powder or a viscus liquid for processing into tablets or capsules.

In yet another embodiment of the invention, 17-AAG is formulated into a liquid or semi-solid capsule for oral delivery. 17-AAG can be filled into capsules as a suspension in a suitable and compatible organic solvent that is appropriate for oral administration of pharmaceuticals. Examples of such solvents, but not limited to, are listed in TABLE 4. These solvents may be used alone or in combination with other solvents and excipients such as sodium lauryl sulfate. The 17-AAG may be loaded as a solution or suspension into such solvents under mild heat at which the drug/excipient exists as a flowable liquid which is subsequently filled into capsules. Upon cooling, the drug/excipient mixture solidifies. The drug may be formulated as a room temperature liquid that is filled into capsules. The 17-AAG active pharmaceutical ingredient can be micronized prior to use in liquid or semi-solid dosage capsule formulations.

In one embodiment 17-AAG is formulated for a capsule dosage form, with Neusilin and sodium lauryl sulfate in a formulation of 33% 17-AAG, 64% Neusilin, and 3% SLS. 17-AAG is first mixed with Neusilin in 1 to 2 (w/w) ratio, the powder mixture is then ground to form a uniform well mixed powder, then sodium lauryl sulfate is added to the powder blend and mixed. The mixture is used to fill a gel capsule for oral administration.

Potential formulations along this line can contain as much as 70% 17-AAG. Other potential options are formulations containing as much as 30% SLS. These powder formulations can also contain other excipients such as lubricants (e.g., magnesium stearate, stearic acid), glidants (e.g., silicon dioxide), secondary fillers (e.g., lactose, microcrystalline cellulose), wetting agents (e.g., Tween 80, PEG3340), polymeric binders (e.g., polyvinyl pyrolidone), and disintegrants (e.g., crosscarmellose sodium). This powder formulation can be filled into a capsule either as a dry blend, dry granulate or wet granulate. This powder formulation can also be compressed as a tablet either by direct compression, dry granulation, or wet granulation process.

The invention further contemplates delayed/sustained release dosage forms. The tablets or gel caplets may be coated by known techniques to delay disintegration and absorption in the gastrointestinal tract and thereby provide a sustained action over a longer period. An example would to be enteric coat the tablet to delay release until the dosage form enters the intestinal tract. The product can be formulated with bio-erodable polymers and swellable polymers or combination thereof or employing a time delay material such as ethyl cellulose or cellulose acetate butyrate. The formulated powder can be wet granulated, extruded, and spherinized to different sizes. The different sized spherinized products can be mixed in various proportions to obtain the desired release characteristics. The spheres can be subsequently coated with a waxy excipient (e.g., Caranuba wax) to modify the dissolution characteristics versus non coated spheres.

The approaches described above can applied to preparing a layered tablet product. As an example, a layered tablet formulation can be prepared with a more rapidly dissolving outer layer and a slow dissolving inner core.

### III. Characterization and Evaluation of the Drug Formulations

A. Stability Determination of the Active Ingredient Using HPLC

The chemical stability of the active therapeutical ingredient, e.g., 17-AAG, can be assessed by HPLC. Specific assay procedures can be developed that allow for the separation of the therapeutically active ansamycin from its degradation products. The extent of degradation can be assessed either from the decrease in signal in the HPLC peak associated with the therapeutically active ansamycins and/or by the increase in signal in the HPLC peak(s) associated with degradation products. Ansamycins, relative to other components of the formulation, are easily and quite specifically detected at their absorbance maximum of 345 nm.

B. Characterization and Assessment of Chemical and Physical Stability of the Phospholipids

Phospholipids and degradation products may be determined after being extracted from dispersions/suspensions. The lipid mixture can then be separated in a two-dimensional thin-layer chromatographic (TLC) system or in a high performance liquid chromatographic (HPLC) system. In TLC, spots corresponding to single constituents can be removed and assayed for phosphorus. Total phosphorous in a sample can be quantitatively determined, e.g., by a procedure using a spectrophotometer to measure the intensity of blue color developed at 825 nm against water. In HPLC, phosphatidylcholine (PC) and phosphatidylglycerol (PG) can be separated and quantified with accuracy and precision.

Lipids can be detected in the region of 203-205 nm. Unsaturated fatty acids exhibit high absorbance, while the saturated fatty acids exhibit lower absorbance in the 200 nm wavelength region of the UV spectrum. As an example, Vemuri and Rhodes, supra, described the separation of egg yolk PC and PG on Licrosorb Diol and Licrosorb S 1-60. The separations used a mobile phase of acetonitrile-methanol with 1% hexane-water (74:16:10 v/v/v). In 8 minutes, separation of PG from PC was observed. Retention times were approximately 1.1 and 3.2 min, respectively.

C. Evaluation of the Dispersion

Dispersion visual appearance, average droplet size, and size distribution are important parameters to observe and maintain. There are a number of methods to evaluate these parameters. For example, dynamic light scattering and electron microscopy are two techniques that can be used. (See, e.g., Szoka and Papahadjopoulos, Annu. Rev. Biophys. Bioeng., 1980, 9:467-508 Morphological characterization, in particular, can be accomplished using freeze fracture electron microscopy. Less powerful light microscopes can also be used.

Dispersion droplet size distribution can be determined, e.g., using a particle size distribution analyzer such as the CAPA-500 made by Horiba Limited (Ann Arbor, Mich., USA), Nanatrac (Mierotrac, Largo, Fla., USA), Coulter Counter (Beckman Coulter Inc., Brea, Calif., USA), or a Zetasizer (Malvern Instruments, Southborough, Mass., USA).

### IV. Evaluation of Oral Absorption Potentials

Among the key factors governing the oral absorption of drugs are solubility in gastrointestinal fluids and membrane permeation potential. These factors can be evaluated in vitro for the pharmaceutical active ingredient using simulated intestinal fluid (SIF) as a solubility matrix and a Caco-2 cell monolayer as the biologic membrane in order to assess the potential for oral delivery of this compound.

A. Solubility Assay

The solubility of an active ingredient may be assayed by its dissolution in a simulated intestinal fluid (SIF) (per USP w/o pancreatin). Conforma Therapeutics has developed a database of SIF solubility data using this assay for a structurally diverse set of compounds that have also been administered orally to mice. Based upon this data, SIF solubility values in excess of 80 μg/mL have been associated with good oral absorption in mice.

For the solubility assay, the low melt form of 17-AAG was mixed with a simulated intestinal fluid to achieve a final known concentration, e.g. 500 μg/mL, in a siliconized tube. The mixture was incubated with continuous shaking for a predetermined time and temperature, e.g., for 24 hours at 37° C. After the incubation, the supernatant is recovered and the concentration of the 17-AAG in the supernatant is determined using a standardized HPLC-UV assay. The solubility of the active ingredient is calculated as the difference between the initial and final concentration of the active ingredient in the supernatant normalized to the total volume of SIF used. It has been found that the solubility of 17-AAG in SIF was 88 μg/mL

B. Permeability Assay

The permeability of the pharmaceutical active ingredient across the gastrointestinal membrane may be estimated from it permeability across a Caco-2 cell monolayer using standard techniques (e.g. Chong S et al. Pharmaceutical Research 1997 14(12):1835-1837). The reference is hereby expressly incorporated by reference.

The permeability potential of the low melt form of 17-AAG was assayed (see, Example 16). Propranolol was included in each assay as a high permeability control compound. It has been found that 17-AAG has a permeability potential across the Caco-2 monolayer of 1.9×10⁻⁵ cm/sec which was higher than the 1.6×10⁻⁵ cm/sec value observed for the high permeability control compound propranolol.

In view of its acceptable SIF solubility and high permeability, 17-AAG may be considered as a compound having good absorption potential.

### V. In Vivo Characterization

A. Absolute Oral Bioavailability Evaluation of Phospholipid Formulation

The absolute bioavailability of the active pharmaceutical ingredients following oral administration of the oral formulation of the invention can be assessed though an in vivo animal study. A 17-AAG phospholipid oral formulation was selected for the study. See, Example 17.

Female Sprague-Dawley rats were administered a phospholipid complex formulation of the low melt form of 17-AAG by oral gavage or as intravenous infusion (tail vein). Blood samples were collected prior to dosing and up to 6 hrs post dosing. The serum concentrations of 17-AAG and the active metabolite 17-aminogeldanamycin (17-AG) were determined using a standardized LC/MS method. The area under the 17-AAG and 17-AG serum concentration-vs-time curves (AUC) for each animal was determined from 0 to 6 hours using the linear trapezoidal rule. The apparent absolute oral bioavailability (F) of 17-AAG was estimated using the following equation:

% F=[(Oral AUC₍₀₋₆ₕ₎×I.V. Dose)/(I.V. AUC₍₀₋₆ₕ₎×Oral Dose)]*100.

The mean apparent absolute oral bioavailability of 17-AAG following oral administration was found to be 109±63%. Accordingly, administration via oral route achieves similar bioavailability as administration via injection.

B. Absolute Oral Bioavailability Evaluation of a Gel Capsule Dose Form

The oral absorption of 17-AAG following the administration of solid dosage form can be similarly estimated in a rat model. See, Example 18. The dosage form consisted of soft gelatin capsules containing 17-AAG (33% w/w), Neusilin (64% w/w) and SLS (3% w/w). The average dose administered was 14.3 mg/kg. The mean apparent absolute oral bioavailability of 17-AAG following oral administration of the prototype, a soft gelatin capsule, was 13±4%.

C. Mouse Pharmacokinetics After Administering of A 17-AAG Phospholipid Formulation

The bioavailability of the active pharmaceutical ingredients after administration of 17-AAG in a phospholipid formulation can be assessed by determining the serum pharmacokinetics in mice. See, Example 19.

A phospholipid formulation of 17-AAG was prepared and the concentration adjusted so that mice received a dose of 2-15 mL/kg administered intravenously (I.V.) as a bolus, orally, or intra peritoneally (I.P.). Blood was collected at predetermined intervals and the serum was isolated. Serum concentrations of 17-AAG and the active metabolite, 17-AG, were determined by UV absorbance using standard HPLC methods.

The oral bioavailability of 17-AAG (% F) administered in the phospholipid complex was determined by dividing the AUC of 17-AAG administered I.V. by the AUC of 17-AAG administered via the oral or I.P route. The total absorption refers to the amount of 17-AAG and 17-AG absorbed after an oral or IP dose relative to the I.V. dose at the same concentration. Fifty-four percent of the 17-AAG administered was found to be orally bio-available. Sixty-three percent of the 17-AAG administered was found to be orally available as 17-AAG or its metabolite, 17-AG.

D. Mouse Pharmacokinetics After Administering of Low Melt 17-AAG in A PEG-Tween Formulation

The bioavailability of the active pharmaceutical ingredients after administration of 17-AAG in a PEG-Tween formulation can be similarly assessed by determining the serum pharmacokinetics in six to 8 week old Balb/C mice. See, Example 20.

A PEG-Tween formulation of 17-AAG was prepared and the concentration adjusted so that mice received a dose of 2-15 mL/kg administered intravenously (I.V.) as a bolus, orally, or I.P.

The oral bioavailability of 17-AAG (% F) administered in PEG-Tween was determined by dividing the AUC of 17-AAG administered I.V. by the AUC of 17-AAG administered via the oral or I.P route. The total absorption refers to the amount of 17-AAG and 17-AG absorbed after an oral or IP dose relative to the I.V. dose at the same concentration. Twenty-five percent of the 17-AAG administered was found to be orally available as 17-AAG or its metabolite, 17-AG.

### VI. Method of Using the Oral Formulations

A. Dose Range

A phase I pharmacologic study of 17-AAG in adult patients with advanced solid tumors determined a maximum tolerated dose (MTD) of 40 mg/m² when administered daily by 1-hour infusion for 5 days every three weeks. (Wilson et al., Am. Soc. Clin. Oncol., abstract, “Phase I Pharmacologic Study of 17-(Allylamino)-17-Demethoxygeldanamycin (AAG) in Adult Patients with Advanced Solid Tumors” 2001. In this study, mean±SD values for terminal half-life, clearance and steady-state volume were determined to be 2.5±0.5 hours, 41.0±13.5 L/hour, and 86.6±34.6 L/m², respectively. Plasma Cmax levels were determined to be 1860±660 nM and 3170±1310 nM at 40 and 56 mg/m². Using these values as guidance, it is anticipated that the range of useful patient dosages for formulations of the present invention will include between about 0.40 mg/m² and 4000 mg/m² of active ingredient, where m² represents surface area. Standard algorithms exist to convert mg/m² to mg of drug/kg patient bodyweight.

### Examples

The following examples are offered by way of illustration only, and all drugs, components, molar ratios, concentrations, pH and steps included therein are not intended to be limiting of the invention unless specifically recited in the claims. Compound preparations of Examples 1-12 are reproduced appropriately below, from commonly owned U.S. Provisional Application Ser. Nos. 60/371,668 and 60/478,430, and International Application PCT/US03/10533, entitled NOVEL ANSAMYCIN FORMULATIONS AND METHODS FOR PRODUCING AND USING SAME, filed Apr. 4, 2003, and International Application PCT/US 03/1053, entitled DRUG FORMULATIONS HAVING LONG AND MEDIUM CHAIN TRIGLYCERIDES, filed Oct. 4, 2003, and to which this application claims priority.

### Example 1

### Preparation of 17-AAG

To 45.0 g (80.4 mmol) of geldanamycin in 1.45 L of dry THF in a dry 2 L flask was added drop-wise over 30 minutes 36.0 mL (470 mmol) of allyl amine in 50 mL of dry THF. The reaction mixture was stirred at room temperature under nitrogen for 4 hr at which time TLC analysis indicated the reaction was complete [(GDM: bright yellow: Rf=0.40; (5% MeOH-95% CHCl₃); 17-AAG: purple: Rf=0.42 (5% MeOH-95% CHC13)]. The solvent was removed by rotary evaporation and the crude material was slurried in 420 mL of H₂O:EtOH (90:10) at 25° C., filtered and dried at 45° C. for 8 hr to give 40.9 g (66.4 mmol) of 17-AAG as purple crystals (82.6% yield, >98% pure by HPLC monitored at 254 nm). MP 206-212° C. ¹H NMR and HPLC are consistent with the desired product.

### Example 2

### Preparation of a Low Melt Form of 17-AAG

The crude 17-AAG from Example 1 is dissolved in 800 mL of 2-propyl alcohol (isopropanol) at 80° C. and then cooled to room temperature. Filtration followed by drying at 45° C. for 8 hr gives 44.6 g (72.36 mmol) of 17-AAG as purple crystals (90% yield, >99% pure by HPLC monitored at 254 nm). MP=147-153° C. NMR and HPLC are consistent with the desired product.

### Example 3

### Solvant Stability of a Low Melt Form of 17-AAG

The 17-AAG product from Example 2 in 400 mL of H₂O:EtOH (90:10) at 25° C., filter and dry at 45° C. for 8 hr to give 42.4 g (68.6 mmol) of 17-AAG as purple crystals (95% yield, >99% pure by HPLC monitored at 254 nm). MP=147-155° C. ¹ H NMR and HPLC are consistent with the desired product.

### Example 4

### Preparation of Compound 237: A dimer

3,3-diamino-dipropylamine (1.32 g, 9.1 mmol) was added dropwise to a solution of geldanamycin (10 g, 17.83 mmol) in DMSO (200 mL) in a flame-dried flask under N₂ and stirred at room temperature. The reaction mixture was diluted with water after 12 hours. A precipitate was formed and filtered to give the crude product. The crude product was chromatographed by silica chromatography (5% CH₃OH/CH₂Cl₂) to afford the desired dimer as a purple solid. The pure purple product was obtained after flash chromatography (silica gel); yield: 93%; mp 165° C.; ¹H NMR (CDC₁₃) 0.97 (d, J=6.6 Hz, 6H, 2CH₃), 1.0 (d, J=6.6 Hz, 6H, 2CH₃), 1.72 (m, 4H, 2 CH₂), 1.78 (m, 4H, 2CH₂), 1.80 (s, 6H, 2CH₃), 1.85 (m, 2H, 2CH), 2.0 (s, 6H, 2CH₃), 2.4 (dd, J=11 Hz, 2H, 2CH), 2.67 (d, J=15 Hz, 2H, 2CH), 2.63 (t, J=10 HZ, 2H, 2CH), 2.78 (t, J=6.5 Hz, 4H, 2CH₂), 3.26 (s, 6H, 20CH₃), 3.38 (s, 6H, 20CH₃), 3.40 (m, 2H, 2CH), 3.60 (m, 4H, 2CH₂), 3.75 (m, 2H, 2CH), 4.60 (d, J=10 Hz, 2H, 2CH), 4.65 (Bs, 2H, 20H), 4.80 (Bs, 4H, 2NH2), 5.19 (s, 2H, 2CH), 5.83 (t, J=15 Hz, 2H, 2CH═), 5.89 (d, J=10 Hz, 2H, 2CH═), 6.58 (t, J=15 Hz, 2H, 2CH═), 6.94 (d, J=10 Hz, 2H, 2CH═), 7.17 (m, 2H, 2NH), 7.24 (s, 2H, 2CH═), 9.20 (s, 2H, 2N—H); MS (m/z) 1189 (M+H).

The corresponding HCl salt was prepared by the following method: an HCl solution in EtOH (5 ml, 0.12 3N) was added to a solution of compound #237 (1 g as prepared above) in THF (15 ml) and EtOH (50 ml) at room temperature. The reaction mixture was stirred for 10 min. The salt was precipitated, filtered and washed with a large amount of EtOH and dried in vacuo. Alternatively, a “mesylate” salt can be formed using methanesulfonic acid instead of HCl.

### Example 5

### Preparation of Compound 914

To geldanamycin (500 mg, 0.89 mmol) in 10 mL of dioxane was added selenium (IV) dioxide (198 mg, 1.78 mmol) at room temperature. The reaction mixture was heated to 100° C. and stirred for 3 hours. After cooling to room temperature, the solution was diluted with ethyl acetate, washed with water and brine, dried over magnesium sulfate, filtered and evaporated in vacuo. The final pure yellow product was obtained after column chromatography (silica gel); yield: 75%; ¹H NMR (CDCl₃) δ 0.97(d, J=7.OHz, 3H, CH3),1.01(d, J=7.OHz, 3H, CH₃), 1.75(m, 3H, CH₂+CH), 2.04(s, 3H, CH₃), 2.41(d, J=9.9 Hz, 1H, CH₂), 2.53(t, J=9.9 Hz, 1H, CH₂), 2.95(m, 1H, CH), 3.30(m, 2, CH+OH), 3.34(s, 6H, 2CH₃), 3.55(m, 1H, CH), 4.09(m, 1H, CH₂), 4.15(s, 3H, CH₃), 4.25(m, 1H, CH₂), 4.41(d, J=9.8 Hz, 1H, CH), 4.80(bs, 2H, CONH₂), 5.32(s, 1H, CH), 5.88(t, J=10.4 Hz, 1H, CH═), 6.04(d, J=9.7 Hz, 1H, CH═), 6.65(t, J=11.5 Hz, 1H, CH═), 6.95(d, J=11.5 Hz, 1H, CH═), 7.32(s, 1H, CH—Ar), 8.69(s, 1H, NH); MS (m/z) 575.6 (M−1);

### Example 6

### Preparation of Compound 967

To compound #914 (50 mg, 0.05 mmol) in 3 mL of THF was added allylamine (3.5 mg, 0.06 mmol). The reaction mixture was stirred at room temperature for 24 hours. The solvent was removed by rotary evaporation. The pure purple product was obtained after column chromatography (silica gel); yield: 90%; ¹H NMR (CDCl₃) δ0.89(d, J=6.6 Hz, 3H, CH₃), 1.03 (d, J=6.9 Hz, 3H, CH₃), 1.78(m, 1H, CH), 1.82(m, 2H, CH₂), 2.04 (s, 3H, CH₃), 2.37(dd, J=13.7 Hz, 1H, CH₂), 2.65(d, J=13.7 Hz, 1H, CH₂), 2.90(m, 1H, CH), 3.33(s, 3H, CH₃), 3.34(s, 3H, CH₃), 3.45(m, 2H, CH+OH), 3.58(m, 1H, CH), 4.14(m, 3H, CH₂+CH₂), 4.16(m, 1H, CH₂), 4.42(s, 1H, OH), 4.43(d, J=1OHz, 1H, CH), 4.75(bs, 2H, CONH₂), 5.33(m, 2H, CH₂═), 5.35(s, 1H, CH), 5.91(m, 2H, CH=+CH═), 6.09(d, J=9.9 Hz, 1H, CH═), 6.46(t, J=5.8 Hz, 1H, NH), 6.66(t, J=11.6 Hz, 1H, CH═), 6.97(d, J=11.6 Hz, 1H, CH═), 7.30(s, 1H, CH), 9.15(s, 1H, NH).

### Example 7

### Preparation of Compound 956

Compound #956 was prepared by the same method described for compound #967 except that azetidine was used instead of allylamine. The final pure purple product was obtained after column chromatography (silica gel); yield: 89%; ¹H NMR (CDCl₃) δ 0.99 (d, J=6.8 Hz, 3H, CH₃), 1.04 (d, J=6.8 Hz, 3H, CH₃), 1.77 (m, 1H, CH), 1.80 (m, 2H, CH₂), 2.06 (s, 3H, CH₃), 2.26 (m, 1H, CH₂), 2.50(m, 2H, CH₂), 2.67 (d, 1H, CH₂), 2.90 (m, 1H, CH), 3.34 (s, 3H, CH₃), 3.36 (s, 3H, CH₃), 3.48 (m, 2H, OH+CH), 3.60 (t, J=6.8 Hz, 1H, CH), 4.11 (dd, J=12 Hz, J=4.5 Hz, 1H, CH₂), 4.30 (dd, J=12 Hz, J=4.5 Hz, 1H, CH₂), 4.45 (d, J=10.0 Hz, 1H, CH), 4.72 (m, 5H, 2CH₂+OH), 4.78 (bs, 2H, NH₂), 5.37 (s, 1H, CH), 5.89 (t, J=10.5 Hz, 1H, CH═), 6.10 (d, J=10 Hz, 1H, CH═), 6.66 (t, J=12 Hz, 1 H, CH═), 7.00 (d, J=12 Hz, 1H, CH═), 7.17 (s, 1H, CH═), 9.20 (s, 1H, CONH); MS(m/z) 602 (M+1).

### Example 8

### Preparation of Compound 529

A solution of 17-aminogeldanamycin (1 mmol) in EtOAc was treated with Na₂S₂0₄ (0.1 M, 300 ml) at RT. After 2 h, the aqueous layer was extracted twice with EtOAc and the combined organic layers were dried over Na₂SO₄, concentrated under reduce pressure to give 18,21-dihydro-17-aminogeldanamycin as a yellow solid. This solid was dissolved in anhydrous THF and transferred via cannula to a mixture of picolinoyl chloride (1.1 mmol) and MS4A (1.2 g). Two hours later, EtN(i-Pr)₂ (2.5 mmol) was further added to the reaction mixture. After overnight stirring, the reaction mixture was filtered and concentrated under reduce pressure. Water was then added to the residue, which was extracted with EtOAc three times; the combined organic layers were dried over Na₂SO₄ and concentrated under reduce pressure to give the crude product which was purified by flash chromatography to give 17-picolinoyl-aminogeldanamycin, Compound 529, as a yellow solid. Rf=0.52 in 80:15:5 CH₂Cl₂:EtOAc:MeOH. Mp=195-197° C. ¹H NMR (CDCl₃) δ 0.91 (d, 3H), 0.96 (d, 3H), 1.71-1.73 (m, 2H), 1.75-1.79 (m, 4H), 2.04 (s, 3H), 2.70-2.72 (m, 2H), 2.74-2.80 (m, 1H), 3.33-3.35 (m, 7H), 3.46-3.49 (m, 1H), 4.33 (d, 1H), 5.18 (s, 1H), 5.77 (d, 1H), 5.91 (t, 1H), 6.57 (t, 1H), 6.94 (d, 1H), 7.51-7.56 (m, 2H), 7.91 (dt, 1H), 8.23 (d, 1H), 8.69-8.70 (m, 1H), 8.75(s, 1H), 10.51 (s, 1H).

### Example 9

### Preparation of Compound 1046

Compound #1046 was prepared according to the procedure described for compound #529 using 4-chloromethyl-benzoyl chloride instead of picolinoyl chloride. (3.1 g, 81%). Rf=0.45 in 80:15:5 CH₂Cl₂:EtOAc:MeOH. ¹H NMR CDCl₃ δ 0.89 (d, 3H), 0.93 (d, 3H), 1.70 (br s, 2H), 1.79 (br s, 4H), 2.04 (s, 3H), 2.52-2.58 (m, 2H), 2.62-2.63 (m, 1H), 2.76-2.79 (m, 1H), 3.33 (br s, 7H), 3.43-3.45 (m, 1H), 4.33 (d, 1H), 4.64 (s, 2H), 5.17 (s, 1H), 5.76 (d, 1H), 5.92 (t, 1H), 6.57 (t, 1H), 6.94 (d, 1H), 7.49 (s, 1H), 7.55 (d, 2H), 7.91 (d, 2H), 8.46 (s, 1H), 8.77 (s, 1H).

### Example 10

### Preparation of Compound 1059

To a solution of compound #1046 (0.14 g, 0.2 mmol) in THF (5 ml) were added sodium iodide (30 mg, 0.2 mmol) and morpholine (35 μL, 0.4 mmol). The resulting mixture was heated at reflux for 10 h whereupon it was cooled to room temperature, concentrated under reduce pressure and the residue was redissolved in EtOAc (30 ml), washed with water (10 ml), dried with Na₂SO₄ and concentrated again. The residue was then recrystallized in EtOH (10 ml) to give the compound 1059 as a yellow solid (100 mg, 66%). Rf=0.10 in 80:15:5 CH₂Cl₂:EtOAc:MeOH. ¹H NMR CDCl₃ δ 0.93 (s, 3H), 0.95 (d, 3H), 1.70 (br s, 2H), 1.78 (br s, 4H), 2.03 (s, 3H), 2.48 (br s, 4H), 2.55-2.62 (m, 3H), 2.74-2.79 (m, 1H), 3.32 (br s, 7H), 3.45 (m, 1H), 3.59 (s, 2H), 3.72-3.74 (m, 4H), 4.32 (d, 1H), 5.15 (s, 1H), 5.76 (d, 1H), 5.91 (t, 1H), 6.56 (t, 1H), 6.94 (d, 1H), 7.48 (s, 1H), 7.50 (d, 2H), 7.87 (d, 2H), 8.47 (s, 1H), 8.77 (s, 1H).

### Example 11

### Preparation of Compound 1236

Compound #1236 was prepared according to the procedure described for compound #1059 using benzylethyl amine instead of morpholine. Rf=0.43 in 80:15:5 CH₂Cl₂:EtOAc:MeOH. ¹H NMR CDCl₃ δ 0.925 (s, 3H), 0.95 (d, 3H), 1.09 (t, 3H), 1.70 (br s, 2H), 1.79 (br s, 4H), 2.04 (s, 3H), 2.50-2.62 (m, 5H), 2.75-2.79 (m, 1H), 3.32 (br s, 7H), 3.46 (m, 1H), 3.59 (s, 2H), 3.63 (s, 2H), 4.33 (d, 1H), 5.16 (s, 1H), 5.78 (d, 1H), 5.91 (t, 1H), 6.57 (t, 1H), 6.94 (d, 1H), 7.25-7.27 (m, 1H), 7.32-7.38 (m, 4H), 7.48 (s, 1H), 7.53 (d, 2H), 7.85 (d, 2H), 8.47 (s, 1H), 8.77 (s, 1H).

### Example 12

### Preparation of Compound 563: 17-(benzoyl)-aminogeldanamycin

A solution of 17-aminogeldanamycin (1 mmol) in EtOAc was treated with Na₂S₂O₄ (0.1 M, 300 mL) at RT. After 2 h, the aqueous layer was extracted twice with EtOAc and the combined organic layers were dried over Na₂SO₄, concentrated under reduce pressure to give 18,21-dihydro-17aminogeldanamycin as a yellow solid. This solid was dissolved in anhydrous THF and transferred via cannula to a mixture of benzoyl chloride (1.1 mmol) and MS4A (1.2 g). Two hours later, EtN(i-Pr)₂ (2.5 mmol) was further added to the reaction mixture. After overnight stirring, the reaction mixture was filtered and concentrated under reduce pressure. Water was then added to the residue which was extracted with EtOAc three times, the combined organic layers were dried over Na₂SO₄ and concentrated under reduce pressure to give the crude product which was purified by flash chromatography to give 17-(benzoyl)-aminogeldanamycin. Rf=0.50 in 80:15:5 CH₂C₁₂:EtOAc:MeOH. Mp=218-220° C. ¹H NMR (CDC₁₃) 0.94 (t, 6H), 1.70 (br s, 2H), 1.79 (br s, 4H), 2.03 (s, 3H), 2.56 (dd, 1H), 2.64 (dd, 1H), 2.76-2.79 (m, 1H), 3.33 (br s, 7H), 3.44-3.46 (m, 1H), 4.325 (d, 1H), 5.16 (s, 1H), 5.77 (d, 1H), 5.91 (t, 1H), 6.57 (t, 1H), 6.94 (d, 1H), 7.48 (s, 1H), 7.52 (t, 2H), 7.62 (t, 1H), 7.91 (d, 2H), 8.47 (s, 1H), 8.77 (s, 1H).

### Example 13

### Preparation of a 17-AAG Aqueous Phospholipid Dispersion

17-AAG was formulated as a 1% (w/w) aqueous phospholipid dispersion. L-histidine, and sucrose are dissolved in water. The phospholipids are added and a high-shear mixer is used to disperse the phospholipids for five minutes at about 3500 rpm. 17-AAG is added to the phospholipid dispersion and mixed with the high-shear mixer to mix/disperse 17-AAG in the phospholipids. The product is removed from the high shear mixer, then slowly mixed (no vortex) to allow the product to allow most of the entrained air to escape. The 0.7 g of a 50/50 (w/w) mixture of ethanol and Tween 80 are added to the stirring 17-AAG dispersion and mixed for one hour to allow more entrained air to escape. The 17-AAG dispersion is microfluidized at 16-19,000 psi to reduce the particle size of the dispersion from about 5 μm to 0.1-0.5 μm (mean particle size). The formulation composition is below:

| Ingredient | % by weight | Function |
|---|---|---|
| 17-AAG | 1.0 | Active Ingredient |
| L-histidine | 0.1 | Buffer |
| Sucrose | 7.5 | tonicity/cryoprotectant |
| Phospholipid | 18.8 | lipid complex/carrier |
| Ethanol | 0.5 | process aid |
| Tween 80 | 0.5 | process aid |
| Water | 71.5 | Diluent |

### Example 14

### Preparation of a 17-AAG Dispersion in 8/2 PEG400/Tween 80

In a 20 mL vials, 8.0 g of PEG400 and 2.0 g of Tween 80 were added and mixed. In a separate 20 mL vial, 100 mg of 17-AAG was added followed by 9.9 g of the 8/2 (w/w) PEG400/Tween 80. The sample was mixed for about one hour with intermittent sonication to dissolve the 17-AAG, resulting in a deep purple viscous solution.

### Example 15

### Lyophilization

Illustrative lyophilization schemes that can be used are described in the following Table.

| Initial | Final | | |
|---|---|---|---|
| Temp. | Temp. | Pressure | |
| (° C.) | (° C.) | (mTorr) | Action |
| 25 | −40 | Ambient | Ramp at 1° C./min |
| −40 | −40 | Ambient | Hold for 60 min |
| −40 | −40 | 50 | Condenser at |
| | | | −60° C. to −80° C. |
| −40 | −28 | | Ramp at 1° C./min |
| −28 | −28 | 50 | Hold for 7200 min |
| −28 | 30 | 50 | Ramp at 1° C./min |
| 30 | 30 | 50 | Hold for 300 min |

Complete
Stopper vials under N₂ at approximately 0.9 atm

### Example 16

### Evaluation of the Oral Absorption Potential of 17-AAG

Among the key factors governing the oral absorption of drugs are solubility in gastrointestinal fluids and membrane permeation potential. These factors were evaluated for 17-AAG using simulated intestinal fluid (SIF) as a solubility matrix and a Caco-2 cell monolayer as the biologic membrane in order to assess the potential for oral delivery of this compound.

For the solubility assay, 17-AAG was weighed into a siliconized tube and simulated intestinal fluid (per USP w/o pancreatin) was added to achieve a final concentration of 500 μg/mL. The tube was then sonicated for 5 minutes then incubated for 24 hours at 37° C. with shaking. The tube was then centrifuged at 20,000×G for 20 minutes and the 17-AAG concentration of the supernatant determined using a standardized HPLC-UV assay. Conforma Therapeutics has developed a database of SIF solubility data using this assay for a structurally diverse set of compounds that have also been administered orally to mice. Based upon this data, SIF solubility values in excess of 80 μg/mL have been associated with good oral absorption in mice.

The permeability of 17-AAG at a concentration of 10 μM was assessed across a Caco-2 cell monolayer in three separate experiments using standard techniques (e.g. Chong S et al. Pharmaceutical Research 1997 14: (12):1835-1837). Propranolol was included in each assay as a high permeability control compound.

The solubility of 17-AAG in SIF was 88 μg/mL and 17-AAG permeability across the Caco-2 monolayer was 1.9×10⁻⁵ cm/sec. The permeability of 17-AAG was in excess of the 1.6×10⁻⁵ cm/sec value observed for the high permeability control compound propranolol. In view of its acceptable SIF solubility and high permeability, 17-AAG was viewed as a compound having good absorption potential.

### Example 17

### Evaluation of Absolute Oral Bioavailability of 17-AAG in an Oral Phospholipid Formulation in Female Rats

The objective of study was to assess the absolute bioavailability of 17-AAG following oral administration of a lipid complex formulation of 17-AAG to rats. Female Sprague-Dawley rats with jugular vein catheters (Charles River Laboratories) were administered the lipid complex formulation of 17-AAG by oral gavage at a dose of 50 mg/kg (n=3) or as two-minute intravenous infusion (tail vein) at a dose of 10 mg/kg (n=3). Blood samples (n=11) were collected via the jugular vein catheter prior to dosing and up to 6 hrs post dosing. The blood was allowed to clot at room temperature for approximately 20 minutes and then placed on ice until centrifugation at 10,000×G for 10 minutes. Serum was collected into polypropylene tubes and frozen at −80° C. until analysis. The serum concentrations of 17-AAG and the active metabolite, 17-AG, were determined using a standardized LC/MS method with a 50 ng/mL limit of quantitation for 17-AAG and 17-AG.

The area under the 17-AAG and 17-AG serum concentration-vs-time curves for each animal was determined from 0 to 6 hours using the linear trapezoidal rule. The apparent absolute oral bioavailability (F) of 17-AAG was estimated using the following equation:

% F=[(Oral AUC₍₀₋₆ₕ₎×I.V. Dose)/(I.V. AUC₍₀₋₆ₕ₎×Oral Dose)]*100.

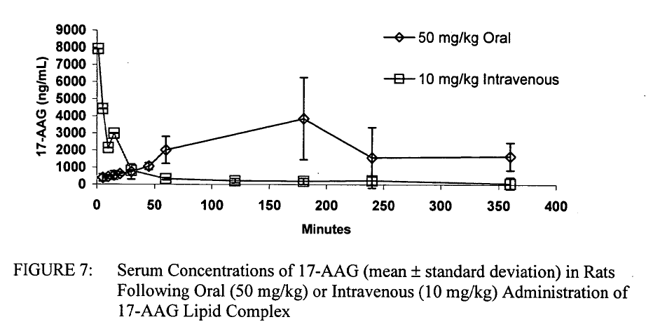

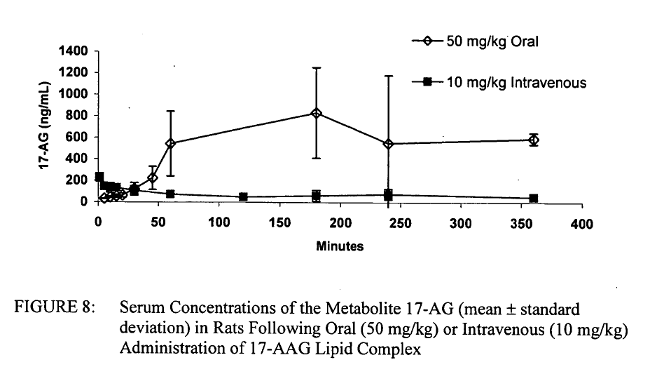

17-AAG was measurable in the serum all animals five minutes following oral administration, reaching a Cₘₐₓ of 3.9±2.4 μg/mL at 1 to 3 hrs (FIG. 7, Table 5). Six hours post oral dosing, the final blood collection time, 17-AAG remained measurable in all animals with a mean concentration of 1.6±0.8 μg/mL. The active metabolite 17-AG was measurable in all animals by 15 minutes after oral dosing, reaching a Cₘₐₓ of 0.9±0.2 μg/mL at 1 to 3 hrs (FIG. 8, Table 5). Six hours post oral dosing, 17-AG remained measurable in all animals with a mean concentration of 0.6±0.1 μg/mL. The mean apparent absolute oral bioavailability of 17-AAG following oral administration was 109±63%.

FIG. 7 shows the serum concentrations of 17-AAG (mean±standard deviation) in rats following oral (50 mg/kg) or intravenous (10 mg/kg) administration of a 17-AAG phospholipide complex

FIG. 8 shows the corresponding serum concentrations of the metabolite 17-AG (mean±standard deviation) in rats following oral (50 mg/kg) or intravenous (10 mg/kg) administration of the 17-AAG phospholipid complex

TABLE 5
Summary of 17-AAG and 17-AG Pharmacokinetic Parameters Following Oral and
Intravenous Administration of 17-AAG Lipid Complex

| | 17- | | | | 17- | | Apparent |
|---|---|---|---|---|---|---|---|
| | AAG | | 17-AAG | 17-AG | AAG | 17-AG | Absolute |
| | Dose | Route of | AUC₍₀₋₆₎ | AUC₍₀₋₆₎ | Cmax | Cmax | Bioavailability |
| Animal # | (mg/kg) | Administration | (μg/mL × min) | (μg/mL × min) | (μg/mL) | (μg/mL) | (%) |
| 1 | 10 | Intravenous | 135 | 37 | 7.0 | 0.2 | — |
| 2 | 10 | Intravenous | 172 | 17 | 8.7 | 0.3 | — |
| 3 | 10 | Intravenous | 161 | 25 | 8.0 | 0.2 | — |
| | | Mean | 156 | 26 | 7.9 | 0.2 | — |
| | | (SD) | (19) | (10) | (0.9) | (0.1) | |
| 4 | 50 | Oral | 1248 | 252 | 5.7 | 1.2 | 160 |
| 5 | 50 | Oral | 1003 | 238 | 4.7 | 0.9 | 129 |
| 6 | 50 | Oral | 305 | 156 | 1.2 | 0.9 | 39 |
| | | Mean | 852 | 215 | 3.9 | 1.0 | 109 |
| | | (SD) | (489) | (52) | (2.4) | (0.2) | (63) |

### Example 18

### Oral Bioavailability of Prototype 17-AAG Solid Dosage Form in Female Rats

The objective of this study was to assess the oral absorption of 17-AAG following administration of a prototype solid dosage form to rats. The dosage form consisted of soft gelatin capsules containing 17-AAG (33% w/w), Neusilin (64% w/w) and SLS (3% w/w). Female Sprague-Dawley rats (n=3) with jugular vein catheters (Charles River Laboratories) were administered a single capsule containing 4.5 mg 17-AAG by oral gavage. The average dose administered was 14.3 mg/kg. Blood samples (n=11) were collected via the jugular vein catheter prior to dosing and up to 8 hrs post dosing. The blood was allowed to clot at room temperature for approximately 20 minutes and then placed on ice until centrifugation at 10,000×G for 10 minutes. Serum was collected into polypropylene tubes and frozen at −80° C. until analysis. The serum concentrations of 17-AAG and the active metabolite 17-AG were determined using a standardized LC/MS method with a 50 ng/mL limit of quantitation for 17-AAG and 17-AG.

The area under the 17-AAG serum concentration-vs-time curves for each animal was determined from 0 to 8 hours using the linear trapezoidal rule. The apparent absolute oral bioavailability (F) of 17-AAG was estimated using the following equation:

% F=[(Oral AUC₍₀₋₆ₕ₎×I.V. Dose)/(I.V. AUC₍₀₋₆ₕ₎×Oral Dose)]*100

Intravenous data from a previous rat study Example 8 was used to estimate absolute bioavailability of 17-AAG upon administration of the prototype solid dosage form.

17-AAG was quantifiable in all animals by 60 minutes post dosing, reaching a Cₘₐₓ of 0.139±0.021 μg/mL at 3 to 6 hrs. The active metabolite 17-AG reached a Cₘₐₓ of 0.061±0.009 μg/mL at 3 to 6 hrs.

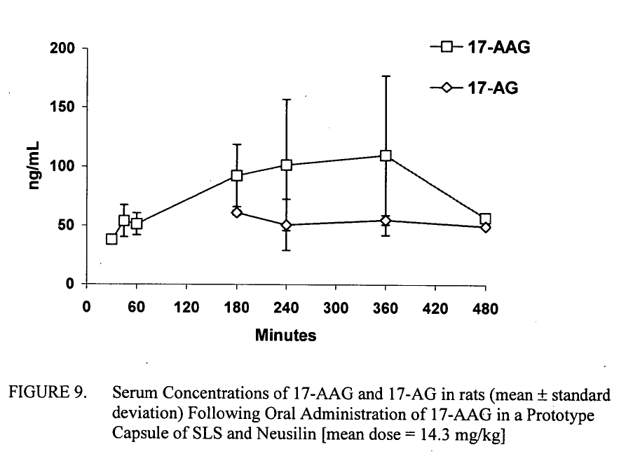

FIG. 9 shows the serum concentrations of 17-AAG and 17-AG in rats (mean±standard deviation) following oral administration of 17-AAG in a prototype capsule [mean dose=14.3 mg/kg]

The mean apparent absolute oral bioavailability of 17-AAG following oral administration of the prototype capsule was 13±4%.

TABLE 6
Summary of 17-AAG and 17-AG Pharmacokinetic Parameters Following Oral
Administration of Prototype 17-AAG Solid Dosage Form (Capsule)

| | 17- | | | | | | Apparent |
|---|---|---|---|---|---|---|---|
| | AAG | | 17-AAG | 17-AG | 17-AAG | 17-AG | Absolute |
| | Dose | Route of | AUC₍₀₋₆₎^{b} | AUC₍₀₋₆₎^{b} | Cmax | Cmax | Bioavailability |
| Animal # | (mg/kg) | Administration | (μg/mL × min) | (μg/mL × min) | (μg/mL) | (μg/mL) | (%) |
| 1^{a} | 10 | Intravenous | 135 | 37 | 7.0 | 0.2 | — |
| 2^{a} | 10 | Intravenous | 172 | 17 | 8.7 | 0.3 | — |
| 3^{a} | 10 | Intravenous | 161 | 25 | 8.0 | 0.2 | — |
| | | Mean | 156 | 26 | 7.9 | 0.2 | — |
| | | (SD) | (19) | (10) | (0.9) | (0.1) | |
| 1 | 13.6 | Oral Capsule | 32 | ND | 0.158 | 0.070 | 15 |
| 2 | 15.1 | Oral Capsule | 19 | ND | 0.117 | 0.061 | 8 |
| 3 | 14.1 | Oral Capsule | 34 | ND | 0.141 | 0.052 | 16 |
| | | Mean | 28 | — | 0.139 | 0.061 | 13 |
| | | (SD) | (8) | | (.021) | (.009) | (4) |

ND = this parameter could not be estimated in this study
^{a}From Example 8
^{b}AUC was estimated from 0 to 8 hrs

### Example 19

### Mouse Pharmacokinetics of Low Melt Form of 17-AAG

The serum pharmacokinetics of the low melt form of 17-AAG were determined in mice in order to assess the bioavailability of the compound after oral administration. Six to 8 week old Balb/C mice were obtained from Harlan Sprague Dawley (Indianapolis, Ind.). The mice were maintained in sterilized filter topped cages or ventilated caging in a room with a 12 h light/12 h dark photoperiod at temperature of 21-23° C. and a relative humidity of 50±5%. Irradiated pelleted food (Harlan Teklad #7912) and autoclaved deionized water were provided ad libitum. Experiments were carried out under institutional guidelines for the proper and humane use of animals in research established by the Institute for Laboratory Animal Research (ILAR).

Drug solutions were prepared and the concentrations were adjusted so that mice received a dose of 2-15 ml/kg administered intravenously (I.V.) as a bolus, orally, or intra-peritoneally (I.P.). For oral administration, 17-AAG was formulated in phospholipids. In I.V. studies blood was sampled at 1, 5, 15, 30, 90 and 120 min. In oral and I.P. studies blood was sampled at 5, 15, 30, 90, 180 minutes and in some cases up to 240 minutes. Blood was collected in capillary tubes, transferred to microcentrifuge tubes, allowed to clot and centrifuged at 14,000 g for 10 minutes at 4° C. to obtain serum. Serum concentrations of 17-AAG and its metabolite, 17-AG, were determined by UV absorbance using standard HPLC methods.

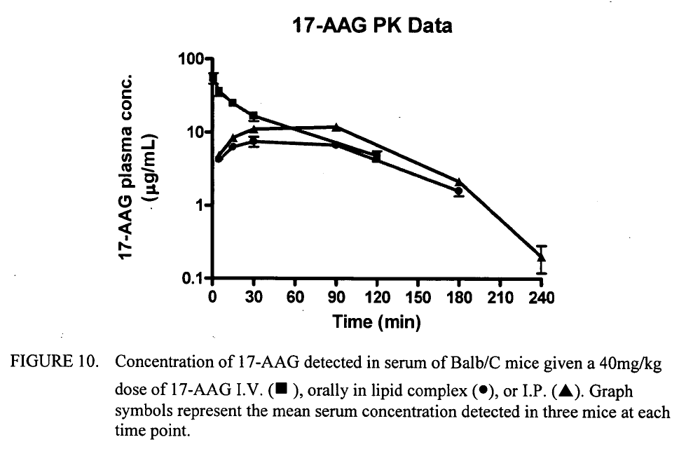

FIG. 10 shows the concentration of 17-AAG detected in serum of Balb/C mice given a 40 mg/kg dose of 17-AAG I.V. (▪), orally in phospholipid complex (), or I.P. (▴). Graph symbols represent the mean serum concentration detected in three mice at each time point.

The pharmacokinetic parameters were determined following the experiment and are shown in the Table 7 and the oral bioavailability is summarized in Table 8 below.

The oral bioavailability of 17-AAG (% F) administered in the lipid complex was determined by dividing the AUC of 17-AAG administered I.V. by the AUC of 17-AAG administered via the oral or I.P route. The total absorption refers to the amount of 17-AAG and 17-AG absorbed after an oral or IP dose relative to the I.V. dose at the same concentration. Fifty-four percent of the 17-AAG administered was found to be orally bio-available. Sixty-three percent of the 17-AAG administered was found to be orally available as 17-AAG or its metabolite, 17-AG.

TABLE 7
Serum Pharmacokinetic Parameter of Low Melt Form of 17-AAG
in Phospholipids - a Mouse Model

| | | | | 17-AAG | | 17-AG |
|---|---|---|---|---|---|---|
| | | Actual | 17-AAG | AUC_{obs} | 17-AG | AUC_{obs} |
| Com- | | Dose | Cₘₐₓ | (μg/mL * | Cₘₐₓ | (μg/mL * |
| pound | Route | (mg/kg) | (μg/mL) | min) | (μg/mL) | min) |
| 17-AAG | IV | 40 | 54.5 | 1752 | 6.2 | 595 |
| 17-AAG | PO | 40 | 7.5 | 953 | 4.5 | 532 |
| 17-AAG | IP | 40 | 11.8 | 1596 | 5.8 | 655 |

TABLE 8
Oral Bioavailability of 17-AAG After Administration of Low Melt
17-AAG in Phospholipid

| | | | Actual | | |
|---|---|---|---|---|---|
| | | | Dose | | Total |
| Compound | Vehicle | Route | (mg/kg) | % F | Absorption |
| 17-AAG | PET | IV | 40 | | |
| 17-AAG | lipid complex | PO | 40 | 54% | 63% |
| 17-AAG | PMSE | IP | 40 | 91% | 96% |

### Example 20

### Mouse Pharmacokinetics PEG-Tween

The serum pharmacokinetics of 17-AAG were determined in mice in order to assess the bioavailability of the compound after oral administration. Six to 8 week old Balb/C mice were obtained from Harlan Sprague Dawley, (Indianapolis, Ind.). The mice were maintained in sterilized filter topped cages or ventilated caging in a room with a 12-h light/12-h dark photoperiod at temperature of 21-23° C. and a relative humidity of 50±5%. Irradiated pelleted food (Harlan Teklad #7912) and autoclaved deionized water were provided ad libitum. Experiments were carried out under institutional guidelines for the proper and humane use of animals in research established by the Institute for Laboratory Animal Research (ILAR).

Drug solutions were prepared and the concentrations were adjusted so that mice received a dose of 2-15 ml/kg administered intravenously (I.V.) as a bolus, orally, or intra-peritoneally (I.P.). In I.V. studies blood was sampled at 1, 5, 15, 30, 90 and 120 min. In oral and I.P. studies blood was sampled at 5, 15, 30, 90, 180 minutes and in some cases up to 240 minutes. Blood was collected in capillary tubes, transferred to microcentrifuge tubes, allowed to clot and centrifuged at 14,000 g for 10 minutes at 4° C. to obtain serum. Serum concentrations of 17-AAG and metabolite were determined by UV absorbance using standard HPLC methods.

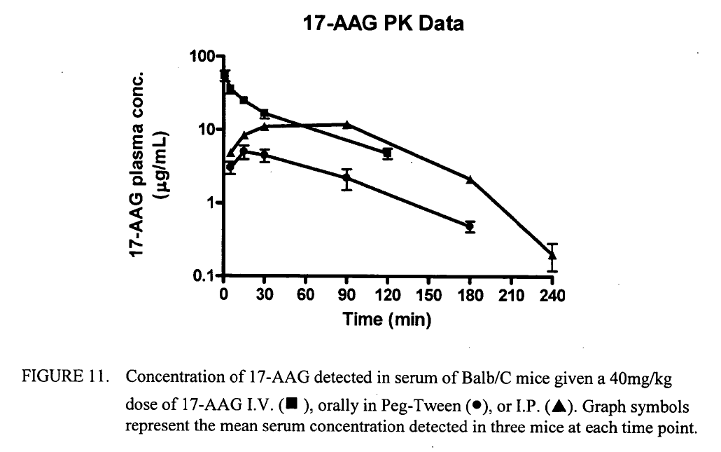

FIG. 11 shows the concentration of 17-AAG detected in serum of Balb/C mice given a 40 mg/kg dose of 17-AAG I.V. (▪), orally in PEG-Tween (), or I.P. (▴). Graph symbols represent the mean serum concentration detected in three mice at each time point.

Pharmacokinetic parameters were determined following the experiment and are shown in Table 9 and the bioavailability calculations were summarized in Table 10 below.

The oral bioavailability of 17-AAG (% F) administered in PEG-Tween was determined by dividing the AUC of 17-AAG administered I.V. by the AUC of 17-AAG administered via the oral or I.P route. The total absorption refers to the amount of 17-AAG and 17-AG absorbed after an oral or IP dose relative to the I.V. dose at the same concentration. Twenty-five percent of the 17-AAG administered was found to be orally available as 17-AAG or its metabolite, 17-AG.

TABLE 9
The Serum Pharmacokinetic Parameter of a Low Melt Form of
17-AAG in PEG-Tween -- a Mouse Model

| | | | | 17-AAG | | 17-AG |
|---|---|---|---|---|---|---|
| | | Actual | 17-AAG | AUC_{obs} | 17-AG | AUC_{obs} |
| Com- | | Dose | Cₘₐₓ | (μg/mL * | Cₘₐₓ | (μg/mL * |
| pound | Route | (mg/kg) | (μg/mL) | min) | (μg/mL) | min) |
| 17-AAG | IV | 40 | 54.5 | 1752 | 6.2 | 595 |
| 17-AAG | PO | 40 | 5.0 | 433 | 2.4 | 163 |
| 17-AAG | IP | 40 | 11.8 | 1596 | 5.8 | 655 |

TABLE 10
Oral Bioavailability of 17-AAG After Administration of a
Low Melt 17-AAG in PEG-Tween

| | | | Actual | | |
|---|---|---|---|---|---|
| | | | Dose | | Total |
| Compound | Vehicle | Route | (mg/kg) | % F | Absorption |
| 17-AAG | PET | IV | 40 | | |
| 17-AAG | PEG-Tween | PO | 40 | 25% | 25% |
| 17-AAG | PMSE | IP | 40 | 91% | 96% |

The foregoing examples are not intended to be limiting of and are merely representative of various embodiments of the invention. It will be readily apparent to one skilled in the art that varying substitutions and modifications may be made to the invention without departing from the scope and spirit of the invention. Thus, such additional embodiments are within the scope of the invention and the following claims.

The reagents described herein are either commercially available, e.g., from Sigma-Aldrich, or else readily producible without undue experimentation using routine procedures known to those of ordinary skill in the art and/or described in publications herein incorporated by reference.

1.-76. (canceled)

77. A pharmaceutical formulation for oral administration comprising
a pharmaceutically effective amount of 17-AAG,
one or more medium-chain triglycerides,
one or more phospholipids, wherein said one or more phospholipids are present in a concentration of at least 5% w/w of the formulation and of at most 25% w/w of the formulation,
one or more long-chain triglycerides, and
oleic acid.

78. The pharmaceutical formulation for oral administration of claim 77 further comprising one or more excipients selected from lubricants, glidants, fillers, wetting agents, binders, disintegrants, flavoring agents, and suspending agents.

79. The pharmaceutical formulation for oral administration of claim 77 wherein said 17-AAG is characterized by a DSC melting temperature at about 156° C.

80. The pharmaceutical formulation for oral administration of claim 77 having an oral bioavailability greater than 35%.

81. The pharmaceutical formulation for oral administration of claim 80 having an oral bioavailability greater than 50%.

82. The pharmaceutical formulation for oral administration of claim 81
having an oral bioavailability greater than 60%.

83. The pharmaceutical formulation for oral administration of claim 77 wherein said concentration of the phospholipid is from about 10% to about 25% w/w of the formulation.

84. The pharmaceutical formulation for oral administration of claim 77 wherein said phospholipid is a phosphoglyceride, sphinogomyelin or a combination thereof.

85. The pharmaceutical formulation for oral administration of claim 77 wherein said phospholipid comprises one or more members selected from phosphatidylcholine, phosphatidylserine, phosphatidylinositol, phosphatidyl ethanolamine and Phospholipon 90G.

86. The pharmaceutical formulation for oral administration of claim 85
wherein said phospholipid is Phospholipon 90G.
